(19) United States
(12) Patent Application Publication
(10) Pub. No.: US 2025/0132055 A1
(43) Pub. Date: Apr. 24, 2025

(54) PARALLEL CANCER SOURCE OF ORIGIN CLASSIFICATION FOR ORGAN TYPE AND TUMOR BIOLOGY TYPE

(71) Applicant: GRAIL, Inc., Menlo Park, CA (US)

(72) Inventors: Joerg Bredno, Menlo Park, CA (US); Kathryn Nelson Kurtzman, San Francisco, CA (US); Rita Shaknovich, New York, NY (US)

(21) Appl. No.: 18/919,817

(22) Filed: Oct. 18, 2024

Related U.S. Application Data
(60) Provisional application No. 63/592,072, filed on Oct. 20, 2023.

(51) Int. Cl. G16H 50/70; G16B 30/10; G16H 50/20

(52) U.S. Cl. CPC G16H 50/70; G16B 30/10; G16H 50/20

(57) ABSTRACT

Methods for cancer source of origin (CSO) prediction are disclosed to predict CSO characteristics. The CSO prediction may include the affected organ or organ group and tumor biology. The method for training parallel CSO classifiers includes obtaining training samples derived from subjects with known cancer diagnosis, each training sample comprising methylation sequence reads corresponding to nucleic acid fragments in a biological sample collected from each subject and each known cancer signal origin including a known affected organ or organ group a plurality of organs or organ groups and a known tumor biology from a plurality of tumor biology classes. The method includes generating, for each training sample, a feature vector based on the methylation sequence reads. The method includes generating a first training data set comprising the feature vectors for the training samples and the known organs or organ groups, and training an organ or organ group classifier with the first training data set to predict organ or organ group from the plurality of organs or organ groups based on an input feature vector. The method includes generating a second training data set comprising the feature vectors for the training samples and the known tumor biology classes, and training a tumor biology classifier with the second training data set to predict tumor biology from the plurality of tumor biology classes based on input feature vector.

### CROSS-REFERENCE TO RELATED APPLICATIONS

The present application claims the benefit of and priority to U.S. Provisional Application No. 63/592,072 filed on Oct. 20, 2023, which is incorporated by reference in its entirety.

### BACKGROUND

Cancer is a leading cause of death worldwide. The fatality of cancer is heightened by the fact that cancer is usually detected in later stages, limiting efficacy of treatment options for long-term survival. Current detection methods generally are specific to the cancer, i.e., each type of cancer (breast, lung, colorectal, prostate, etc.) is separately screened for. Accordingly, each screening process is tailored to the specific cancer. For example, mammography scans are utilized in breast cancer detection, whereas colonoscopy or fecal tests have helped with colorectal cancer detection. Each varied screening method is generally not cross-applicable to other cancers. Furthermore, present screening methods are encumbered by low detection rates or high false positive rates. Low detection rates often fail to detect early-stage cancers as the cancers are just developing. A high false positive rate misdiagnoses cancer-free subjects as positive for cancer status. As a result, most screening tests are only practical when they are used to test subjects who have a high risk of developing the screened cancer or have symptoms indicative of the presence of suspected cancers. As such, most screening tests have limited ability to detect cancers in the general population.

Novel research has implicated aberrant DNA methylation in many disease processes, including cancer. DNA methylation plays a role in regulating gene expression and defining tissue differentiation, cellular identity, and/or embryological lineage. Thus, aberrant DNA methylation can create issues in normal gene expression pathways or cellular identity, thereby leading to cancer or other diseases. For example, specific patterns of differentially methylated regions may be useful as molecular markers for various disease states. Detection of these differentially methylated regions may be accomplished through sequencing analysis of cell-free DNA molecules. In general, cell-free DNA molecules are DNA molecules that arise in bodily fluids. These DNA molecules are typically released due to natural cell death, active release by healthy cells, or tumor-derived DNA molecules shed from tumor cells undergoing cell death. Nonetheless, even techniques that detect differentially methylated regions face a number of challenges. Early cancer detection is particularly challenging due to the miniscule ratio of tumor cells to non-cancer cells in the subject. The miniscule ratio may be on the order of 1:1000, 1:10,000, or even 1:100,000. This creates a challenge of detecting the small amounts of cancer “signal” amidst otherwise healthy “noise”, especially when analyzing this signal with an easily accessible sample condition, for example a blood draw to assess presence of cancer signal in blood plasma, for example in cell-free DNA.

Further challenges may arise when providing insight into cancer detected in a subject. For example, a multi-cancer detection test may only provide a binary prediction as to whether the subject has or does not have cancer. Such insight may limit a healthcare provider's ability to proceed with diagnosis of cancer and/or treatment of the subject. Diagnostic workup and treatment options are generally tailored to the particular organ group that is affected and to the tumor biology. As such, there is a need for increased granularity in analytical predictions to better inform a healthcare provider's diagnostic workup options.

The present disclosure is directed to addressing the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The invention(s) described herein this disclosure provide for improvements to cancer detection, diagnosis and treatment, in particular, providing granularity in cancer source of origin (CSO) predictions. The invention(s) described herein encompass training of parallel CSO classifiers to separately predict the organ type affected by cancer and a tumor biology type. Parallel training of the CSO classifiers divides the CSO predictive analyses to avoid confounding of organ type and tumor biology type predictions when only predicting cancer signal origin generally. In some examples, the CSO classifiers are trained with training data sets derived from the same set of training samples. A training data set is generated for each CSO classifier based on, for example, methylation sequencing data for each sample and a known CSO label for the sample that represents clinical truth which is currently only obtained after a diagnostic workup and cancer diagnosis. Training of the CSO classifiers may be in parallel such that each classifier separately and independently learns patterns in the methylation sequencing data to discriminate organ type and to discriminate tumor biology type, respectively. Accordingly, the parallel-trained CSO classifiers provide additional granularity in CSO predictions, better informing workup steps that yield to a diagnosis of cancer after a screening or early detection has detected cancer signal for example in blood plasma. Moreover, training of the separate classifiers with the same base data avoids the need to perform multiple sequencing assays, thereby also improving the assaying process.

Clause 1. A method for training independent parallel cancer signal origin (CSO) classifiers, the method comprising: obtaining training samples derived from subjects with a known cancer diagnosis, each training sample comprising methylation sequence reads corresponding to nucleic acid fragments in a biological sample collected from each subject and each known cancer diagnosis including a known organ or organ group of a plurality of organs or organ groups affected and a known tumor biology of a plurality of tumor biology classes; generating, for each training sample, a feature vector based on the methylation sequence reads; generating a first training data set comprising the feature vectors for the training samples and the known organ or organ group; training an organ or organ group classifier with the first training data set to predict an organ or organ group from the plurality of organs or organ groups based on an input feature vector; generating a second training data set comprising the feature vectors for the training samples and the known tumor biology classes; and training a tumor biology classifier with the second training data set to predict tumor biology from the plurality of tumor biology classes based on input feature vector.

Clause 2. The method of any preceding clause, further comprising: extracting, for each training sample, the known organ or organ group and the known tumor biology class from the known cancer diagnosis and clinical information.

Clause 3. The method of any preceding clause, wherein the feature vector is based, at least in part, on methylation features based on the methylation sequence reads.

Clause 4. The method of clause 3, wherein the methylation features include: methylation density at one or more loci, density of hypermethylated sequence reads at one or more loci, density of hypomethylated sequence reads at one or more loci, a count of methylation sequence reads determined to be anomalously methylated at one or more loci, or some combination thereof.

Clause 5. The method of any preceding clause, wherein generating the first training data set comprises excluding information regarding tumor biology.

Clause 6. The method of any preceding clause, wherein generating the second training data set comprises excluding information regarding the affected organ or organ group.

Clause 7. The method of any preceding clause, further comprising: determining, for each feature, information gain in discriminating between the organs or organ groups; identifying discriminatory features for the organ or organ group classifier based on the information gains; and modifying the feature vectors of the first training set to consist of the discriminatory features, wherein the modified feature vectors are used in training of the organ or organ group classifier.

Clause 8. The method of any preceding clause, further comprising: determining, for each feature, information gain in discriminating between the tumor biology classes; identifying discriminatory features for the tumor biology classifier based on the information gains; and modifying the feature vectors of the second training set to consist of the discriminatory features, wherein the modified feature vectors are used in training of the tumor biology classifier.

Clause 9. The method of any preceding clause, wherein the organ or organ group classifier or the tumor biology classifier are machine-learning models.

Clause 10. The method of any preceding clause, further comprising training the organ or organ group classifier and the tumor biology classifier in parallel training processes.

Clause 11. The method of any preceding clause, further comprising training the organ or organ group classifier prior to training the tumor biology classifier.

Clause 12. The method of clause 11, wherein outputs of the organ or organ group classifier are appended to the feature vectors of the second training data set prior to training of the tumor biology classifier.

Clause 13. The method of any preceding clause, further comprising training the tumor biology classifier prior to training the organ or organ group classifier.

Clause 14. The method of clause 13, wherein outputs of the tumor biology classifier are appended to the feature vectors of the first training data set prior to training of the organ or organ group classifier.

Clause 15. The method of any preceding clause, wherein the organs or organ groups include: breast; prostate; lung; head or neck; anus; cervix; ovary or fallopian tubes; uterus; bladder or urothelial; kidney; stomach or esophagus; liver or intrahepatic bile duct; pancreas, extrahepatic bile duct, or gall bladder; colon or rectum; bone or soft tissue; skin; blood, lymphatic system, or bone marrow; thyroid; ambiguous tissue; or some combination thereof.

Clause 16. The method of any preceding clause, wherein the tumor biology classes include: lymphoid neoplasm, myeloid neoplasm, plasma cell neoplasm, neuroendocrine carcinoma or tumor, adenocarcinoma, squamous cell carcinoma and not human-papillomavirus-associated (HPV-associated), HPV-associated carcinoma, hepatocellular carcinoma, neoplasm of Mullerian origin, transitional cell carcinoma, mesenchymal tumor, melanocytic neoplasm, mesothelial neoplasm, other tumor biology, ambiguous tumor biology, or some combination thereof.

Clause 17. A method for predicting cancer signal of origin (CSO), the method comprising: obtaining a test sample derived from a subject, the test sample comprising methylation sequence reads corresponding to nucleic acid fragments in a biological sample collected from the subject; generating, for the test sample, a first feature vector based on the methylation sequence reads associated with a first set of features identified as discriminatory for organ or organ group classification; generating, for the test sample, a second feature vector based on the methylation sequence reads associated with a second set of features identifies as discriminatory for tumor biology classification; applying an organ or organ group classifier to the first feature vectors to predict an organ or organ group of a cancer associated with the test sample from a plurality of organs or organ groups; applying a tumor biology classifier to the second feature vector to predict a tumor biology for the cancer associated with the test sample from a plurality of tumor biology classes; wherein the organ or organ group classifier and the tumor biology classifier are independently trained on training samples derived from subjects with a known cancer diagnosis including a known organ or organ group of a plurality of organs or organ groups affected and a known tumor biology of a plurality of tumor biology classes, each training sample comprising methylation sequence reads corresponding to nucleic acid fragments in a biological sample collected from each subject; and informing a diagnostic workup to diagnose a cancer based on the predicted organ or organ groups and the predicted tumor biology.

Clause 18. The method of clause 17, wherein the organ or organ group classifier and the tumor biology classifier are trained by: generating, for each training sample, a feature vector based on the methylation sequence reads of the training sample; generating a first training data set comprising the feature vectors for the training samples and the known organ or organ group of the known cancer diagnosis; training an organ or organ group classifier with the first training data set to predict an organ or organ group from the plurality of organs or organ groups based on an input feature vector; generating a second training data set comprising the feature vectors for the training samples and the known tumor biology classes of the known cancer diagnosis; and training a tumor biology classifier with the second training data set to predict tumor biology from the plurality of tumor biology classes based on input feature vector.

Clause 19. The method of any of clauses 17-18, wherein generating the first training data set comprises excluding information regarding tumor biology, and wherein generating the second training data set comprises excluding information regarding the affected organ or organ group.

Clause 20. The method of any of clauses 17-19, further comprising: determining, for each feature, information gain in discriminating between the organs or organ groups; identifying discriminatory features for the organ or organ group classifier based on the information gains; and modifying the feature vectors of the first training set to consist of the discriminatory features, wherein the modified feature vectors are used in training of the organ or organ group classifier.

Clause 21. The method of any of clauses 17-20, further comprising: determining, for each feature, information gain in discriminating between the tumor biology classes; identifying discriminatory features for the tumor biology classifier based on the information gains; and modifying the feature vectors of the second training set to consist of the discriminatory features, wherein the modified feature vectors are used in training of the tumor biology classifier.

Clause 22. The method of any of clauses 17-21, wherein the organ or organ group classifier or the tumor biology classifier are machine-learning models.

Clause 23. The method of any of clauses 17-22, further comprising training the organ or organ group classifier and the tumor biology classifier in parallel training processes.

Clause 24. The method of any of clauses 17-23, further comprising training the organ or organ group classifier prior to training the tumor biology classifier.

Clause 25. The method of any of clauses 17-24, further comprising training the tumor biology classifier prior to training the organ or organ group classifier.

Clause 26. The method of any of clauses 17-25, further comprising: prior to applying the organ or organ group classifier, modifying the feature vector according to discriminatory features for the organ or organ group classifier to generate a first reduced feature vector such that the organ or organ group classifier is applied to the first reduced feature vector; and prior to applying the tumor biology classifier, modifying the feature vector according to discriminatory features for the tumor biology classifier to generate a second reduced feature vector such that the tumor biology classifier is applied to the second reduced feature vector.

Clause 27. The method of any of clauses 17-26, wherein informing diagnostic workup of a detected cancer signal comprises identifying one or more diagnostic workup options based on the predicted organ or organ group, the predicted tumor biology, or some combination thereof.

Clause 28. The method of any of clauses 17-27, wherein the organs or organ groups include: breast; prostate; lung; head or neck; anus; cervix; ovary or fallopian tubes; uterus; bladder or urothelial; kidney; stomach or esophagus; liver or intrahepatic bile duct; pancreas, extrahepatic bile duct, or gall bladder; colon or rectum; bone or soft tissue; skin; blood, lymphatic system, or bone marrow; thyroid; ambiguous tissue; or some combination thereof.

Clause 29. The method of any of clauses 17-28, wherein the tumor biology classes include: lymphoid neoplasm, myeloid neoplasm, plasma cell neoplasm, neuroendocrine carcinoma or tumor, adenocarcinoma, squamous cell carcinoma and not human-papillomavirus-associated (HPV-associated), HPV-associated carcinoma, hepatocellular carcinoma, neoplasm of Mullerian origin, transitional cell carcinoma, mesenchymal tumor, melanocytic neoplasm, mesothelial neoplasm, other tumor biology, ambiguous tumor biology, or some combination thereof.

Clause 30. The method of any of clauses 17-29, wherein the subject is previously diagnosed with cancer of unknown origin, wherein informing the diagnostic workup comprises informing the diagnostic workup to refine diagnosis based on the predicted organ or organ group and the predicted tumor biology.

Clause 31. The method of any of clauses 17-30, wherein informing the diagnostic workup comprises: providing a report comprising, for the test sample, a cancer signal detected readout, a cancer signal origin prediction, the predicted organ or organ groups, and the predicted tumor biology.

Clause 32. A non-transitory computer-readable storage medium storing instructions that, when executed by one or more computer processors, cause the one or more computer processors to perform the method of any of clauses 1-31.

Clause 33. A system comprising: one or more computer processors; and the non-transitory computer-readable storage medium of clause 32.

Clause 34. A treatment kit comprising: a collection vessel for collecting a DNA sample from a subject; optionally, one or more reagents for isolating DNA fragments in the DNA sample; optionally, one or more probes targeting one or more genomic loci determined to be indicative of cancer status; and the non-transitory computer-readable storage medium of clause 32.

Clause 35. A method for providing a report for a test sample for a patient to assist with a diagnostic workup of the patient, the report comprising a cancer signal detected readout and a cancer signal origin (CSO) prediction, the CSO prediction comprising a predicted organ or organ groups for the CSO and a predicted tumor biology for the CSO, the method comprising: obtaining the test sample derived from a patient, the test sample comprising methylation sequence reads corresponding to nucleic acid fragments in a biological sample collected from the patient; generating, for the test sample, a first feature vector based on methylation information selected to be informative of a cancer signal associated with the test sample; generating, for the test sample, a second feature vector based on the methylation sequence reads associated with a first set of features identified as discriminatory for organ or organ group classification; generating, for the test sample, a third feature vector based on the methylation sequence reads associated with a second set of features identifies as discriminatory for tumor biology classification; applying a cancer signal classifier to the first feature vector to predict the cancer signal of a cancer associated with the test sample; applying an organ or organ group classifier to the second feature vector to predict an organ or organ group of the cancer associated with the test sample from a plurality of organs or organ groups; applying a tumor biology classifier to the third feature vector to predict a tumor biology for the cancer associated with the test sample from a plurality of tumor biology classes; wherein the cancer signal classifier is trained on training samples derived from a plurality of cancer-positive subjects and cancer-negative subjects, each cancer-positive subject having a labeled cancer diagnosis and each cancer-negative subject known to not have cancer, each training sample comprising methylation sequence reads corresponding to nucleic acid fragments in a biological sample collected from each subject; wherein the organ or organ group classifier and the tumor biology classifier are independently trained on training samples derived from subjects with a known cancer diagnosis including a known organ or organ group of a plurality of organs or organ groups affected and a known tumor biology of a plurality of tumor biology classes, each training sample comprising methylation sequence reads corresponding to nucleic acid fragments in a biological sample collected from each subject; generating the report for the test sample comprising the cancer signal detected readout and the cancer signal origin (CSO) prediction based on results from the cancer signal classifier, the organ or organ type classifier, and the tumor biology classifier, the CSO prediction comprising the predicted organ or organ groups for the CSO and the predicted tumor biology for the CSO; and providing the report to the patient or a health care provider for the patient.

Clause 36. A method for providing a report for a test sample for a patient to assist with a diagnostic workup of the patient, the report comprising a cancer signal detected readout and a cancer signal origin (CSO) prediction, the CSO prediction comprising a predicted organ or organ groups for the CSO and a predicted tumor biology for the CSO, wherein the CSO prediction is determined by: obtaining the test sample derived from a patient, the test sample comprising methylation sequence reads corresponding to nucleic acid fragments in a biological sample collected from the patient; generating, for the test sample, a first feature vector based on methylation information selected to be informative of a cancer signal associated with the test sample; generating, for the test sample, a second feature vector based on the methylation sequence reads associated with a first set of features identified as discriminatory for organ or organ group classification; generating, for the test sample, a third feature vector based on the methylation sequence reads associated with a second set of features identifies as discriminatory for tumor biology classification; applying a cancer signal classifier to the first feature vector to predict the cancer signal of a cancer associated with the test sample; applying an organ or organ group classifier to the second feature vector to predict an organ or organ group of the cancer associated with the test sample from a plurality of organs or organ groups; applying a tumor biology classifier to the third feature vector to predict a tumor biology for the cancer associated with the test sample from a plurality of tumor biology classes; wherein the cancer signal classifier is trained on training samples derived from a plurality of cancer-positive subjects and cancer-negative subjects, each cancer-positive subject having a labeled cancer diagnosis and each cancer-negative subject known to not have cancer, each training sample comprising methylation sequence reads corresponding to nucleic acid fragments in a biological sample collected from each subject; wherein the organ or organ group classifier and the tumor biology classifier are independently trained on training samples derived from subjects with a known cancer diagnosis including a known organ or organ group of a plurality of organs or organ groups affected and a known tumor biology of a plurality of tumor biology classes, each training sample comprising methylation sequence reads corresponding to nucleic acid fragments in a biological sample collected from each subject; generating the report for the test sample comprising the cancer signal detected readout and the cancer signal origin (CSO) prediction based on results from the cancer signal classifier, the organ or organ type classifier, and the tumor biology classifier, the CSO prediction comprising the predicted organ or organ groups for the CSO and the predicted tumor biology for the CSO; and providing the report to the patient or a health care provider for the patient.

### BRIEF DESCRIPTION OF DRAWINGS

FIG. 1 is an exemplary flowchart describing an overall workflow of cancer classification of a sample, according to one or more embodiments.

FIG. 2A is an exemplary flowchart describing a process of sequencing a fragment of cell-free (cf) DNA to obtain a methylation state vector, according to one or more embodiments.

FIG. 2B is an exemplary illustration of the process of FIG. 2A of sequencing a fragment of cell-free (cf) DNA to obtain a methylation state vector, according to one or more embodiments.

FIG. 3A is an exemplary flowchart describing a process of generating a control group data structure for determining anomalously methylated fragments, according to one or more embodiments.

FIG. 3B is an exemplary flowchart describing a process of determining a fragment to be anomalously methylated based on the control group data structure, according to one or more embodiments.

FIG. 4A is an exemplary flowchart describing a process of training a cancer classifier, according to one or more embodiments.

FIG. 4B illustrates an example generation of feature vectors used for training the cancer classifier, according to one or more embodiments.

FIG. 5A is an exemplary flowchart describing a process of training parallel cancer source of origin (CSO) classifiers, according to one or more embodiments.

FIG. 5B is an exemplary flowchart describing a process of deployment of parallel CSO classifiers, according to one or more embodiments.

FIG. 6A illustrates an exemplary flowchart of devices for sequencing nucleic acid samples according to one or more embodiments.

FIG. 6B is an exemplary block diagram of an analytics system, according to one or more embodiments.

FIG. 7 illustrates two confusion matrices demonstrating the predictive accuracy of an organ type classifier, according to one or more example implementations.

FIG. 8 illustrates two confusion matrices demonstrating the predictive accuracy of a tumor biology type classifier, according to one or more example implementations.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more insight into the context and status of disease progression and availability of more pathways of treatment that may lead to a beneficial outcome; for loved ones, early cancer detection increases the likelihood of not losing friends and family to the disease.

Early detection and classification of cancer can also be obtained when a patient is being worked up for symptoms that can indicate the presence of a cancer, a process that today can be referred to as a “diagnostic odyssey” that causes stress and anxiety for patients and loved ones over sometimes multiple months of uncertainty with a wide variety of cost-intensive medical procedures.

Recently, early cancer detection technology has progressed towards analyzing genetic fragments (e.g., DNA) in a sample from a person to determine if any of those genetic fragments originate from cancer cells. To increase the benefit of early cancer detection, the sample is often a relatively easy to acquire liquid sample from the person, such as blood, saliva, or urine. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. The limits of determinations like this are typical for most cancers—that is, most cancers are only identifiable once they has grown to a sufficient size to be detected by some sort of imaging technology. For many more types of cancer, there is no screening paradigm like mammography available, and even regular doctor visits cannot detect the presence of a growing and advancing cancer. For example, the person having a high risk of breast cancer above might at the same time be at high risk for ovarian cancer, which is undetectable by mammography or any other currently available image-based cancer screening modality.

Cancer detection using analysis of genetic fragments in a patient's, e.g., blood, alleviates this issue. To illustrate, cancer cells will start shedding DNA fragments into a person's bloodstream as soon as they form. This occurs when there are very few of the cancer cells, and before they would be visible with imaging techniques. With the appropriate methods, therefore, a system that analyzes DNA fragments in the bloodstream (so-called “cell-free DNA” or “cfDNA”) could identify cancer presence in a person based on shed cancer DNA fragments, and, more importantly, the system could do so before the cancer is identifiable using more traditional cancer detection techniques, and even more so for cancers where no traditional cancer screening method exists.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation includes the laboratory methods necessary to prepare DNA fragments for sequencing, sequencing is the process of reading the ordered nucleotides in the samples, and data analysis includes processing and analyzing the genetic information in the sequencing data to identify cancer presence.

Such an analysis to identify the presence of cancer and identify its type is further relevant when a patient has been diagnosed with cancer and additional information is needed for prognosis, treatment decision, and for a patient on treatment to identify residual disease, recurrence, or relapse for a patient on treatment when a treatment has not or is no longer able to control cancer growth.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to NGS technology, cancer detection technologies broadly, and early cancer detection, and ultimately cure, more generally.

To illustrate, as an example, problems introduced in (1) sample preparation include DNA sample quality, sample contamination, fragmentation bias, and accurate indexing. Remedying these problems would yield better genetic data for cancer detection.

Similarly, problems introduced in (2) sequencing include, for example, errors in accurate transcribing of fragments (e.g., reading an “adenine (A)” instead of a “cytosine (C)”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. Sequencing data for a single sample can be on the order of hundreds of thousands (up to millions) of sequence reads, amounting to terabytes of data. Training analytical models typically involves collecting and processing thousands (up to tens of thousands or more) of samples with ascertained and labeled clinical cancer status, affected organ or organ group, primary site of cancer, and underlying cancer biology. Effectively and efficiently analyzing that amount of data is both procedurally and computationally demanding. For instance, analyzing NGS sequencing involves several baseline processing steps such as, e.g., aligning reads to one another, aligning and mapping reads to a reference genome, de-duplicating duplicative reads, detecting contamination of a sample, identifying and calling variant genes, identifying and calling abnormally methylated individual genomic sites or regions, generating functional annotations, etc. Performing any of these processes on terabytes (or more) of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, non-informative regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of the large array of sequencing data typically for the analytical models used in early cancer detection enabled by NGS would be an improvement to cancer detection using NGS sequencing. Moreover, such processes as described and elaborated on herein were crafted as a solution to the various hurdles native to NGS technologies, and as such are non-routine and unconventional activity in this technical field.

As a further example, under (3) data analysis, accurate identification of informative DNA from NGS data to identify a cancer presence is another difficult task native to the field of early cancer detection. To be effective, algorithms are sought to compensate for, e.g., errors generated by sample preparation and sequencing, the large scale of genomic and methylation variety present in the population, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed. Furthermore, such techniques are non-routine and unconventional activity in the technical field of endeavor.

One particular challenge arises in providing predictive insight to a healthcare provider based on a sample for a particular patient. A first method of cancer prediction may involve providing a binary prediction to the healthcare provider of whether a test subject has a likelihood of or does not have a likelihood of cancer. Such insight, though informative, falls short in providing further insight into how best to workup the cancer signal detection to come to a cancer diagnosis while avoiding a so-called “diagnostic odyssey.” A second method of cancer prediction may involve providing a multiclass prediction as to a particular cancer signal origin such as a prediction regarding whether the patient's sample is reflective of one of several discrete cancer signal origins, commonly referred to as organ systems or organ types. This insight improves upon the first method, but can lack specificity in characteristics around the cancer signal origin which can be highly informative to the diagnostic workup options to obtain a cancer diagnosis. Lastly, a third method of cancer prediction may involve providing insight into both the cancer source of origin including the organ type affected and the tumor biology type of the cancer. Such insight most comprehensively arms a healthcare provider to provide the optimal workup options to come to a cancer diagnosis after a test predicted or detected a cancer signal. For example, a report generated by systems utilizing computational models or other algorithms trained to generate said insight may predict that a particular subject has a cancer with a signal origin in the stomach tissue (organ type) with an adenocarcinoma biology (tumor biology type). A report generated based on cancer prediction can include information from all three methods of cancer prediction: cancer signal detected (or not detected), cancer signal origin (if cancer signal detected), and additional prediction information such as organ type and/or tumor biology type. Armed with such insight, a healthcare provider can better evaluate diagnostic workup options to tailor diagnostic workup of the detected cancer signal accordingly. In some embodiments, the analytics system may further store a database of diagnostic steps or workup options for each combination of organ type and tumor biology type. The analytics system may provide diagnostic steps and workup recommendations to a healthcare provider based on the predicted organ type and tumor biology type.

### I.A. Cancer Classification Workflow

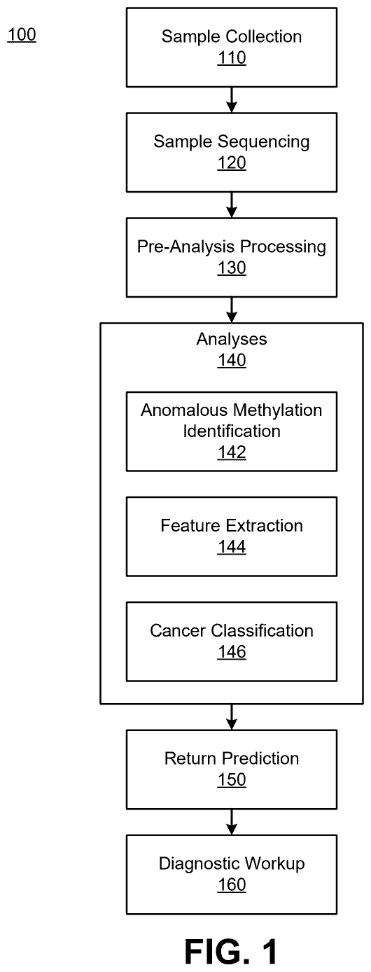

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in subjects. From a healthcare standpoint, the workflow 100 can serve to supplement other existing cancer screening and early detection tools. The workflow 100 may serve to provide early cancer detection and/or routine cancer monitoring, minimal residual disease detection, prognosis, treatment prediction, or subtyping information to better inform treatment plans for subjects diagnosed with cancer. The overall workflow 100 may include additional/fewer steps than those shown in FIG. 1.

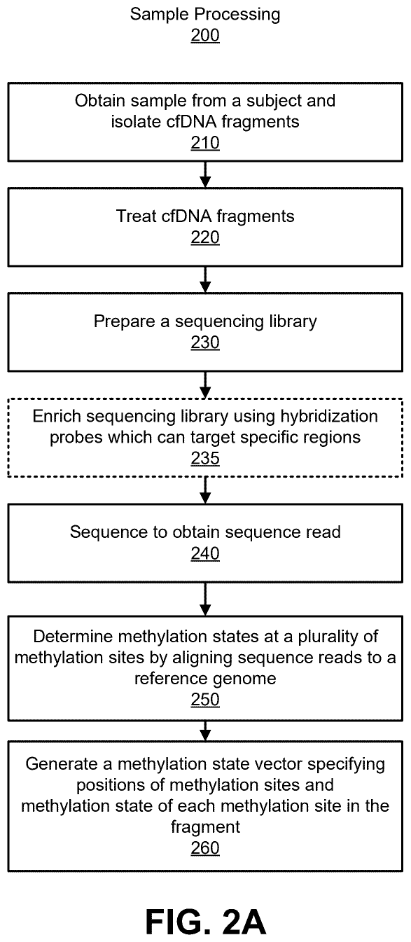

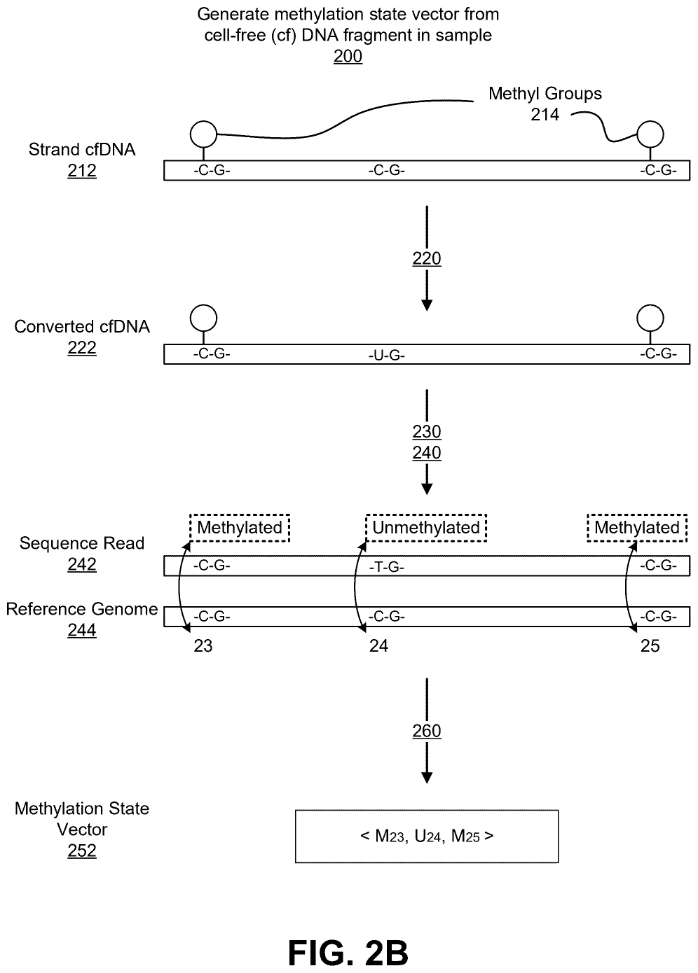

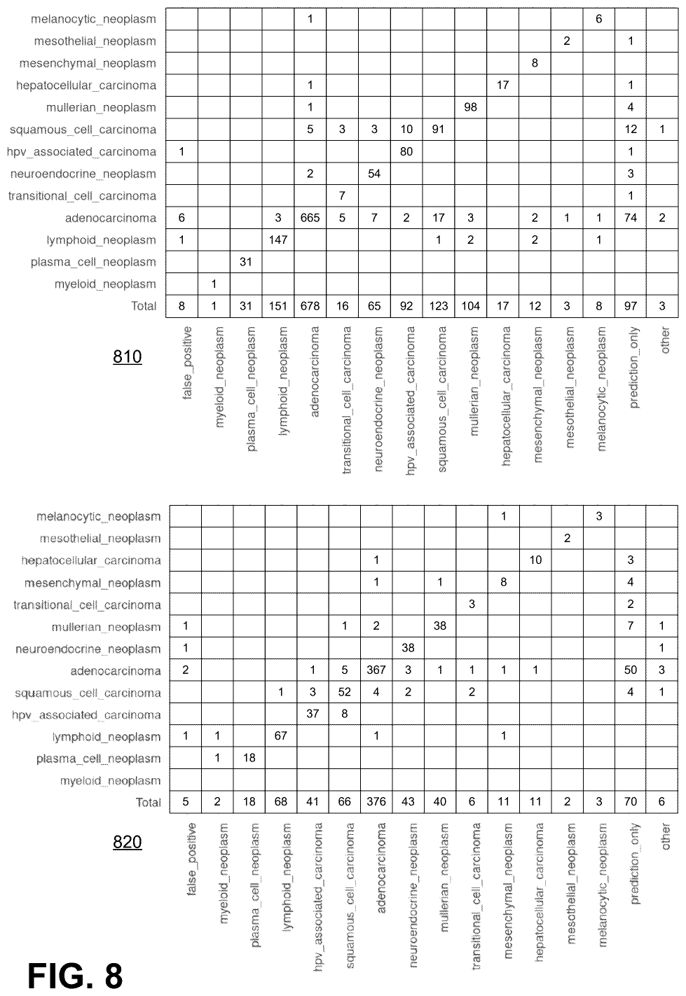

A healthcare provider performs sample collection 110. A subject to undergo screening, early detection or cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the subject, which may be extracted and sequenced for screening, early detection, or cancer classification. Once the sample is collected, the sample is provided to a laboratory process and sequencing device. Along with the sample, the healthcare provider may collect other information relating to the subject, e.g., biological sex, age, race, smoking status, other health metrics, any prior diagnoses, etc. A sequencing device performs sample sequencing 120. A lab clinician may perform one or more processing steps to the sample in preparation of sequencing. Once prepared, the lab technician loads the sample in the sequencing device. An example of devices utilized in sequencing is further described in conjunction with FIGS. 8A & 8B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In context of DNA methylation, bisulfite sequencing (e.g., further described in FIGS. 2A & 2B) can determine methylation status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 8B. Pre-analysis processing 130 may include, but not limited to, de-duplication of sequence reads, determining metrics relating to coverage, determining whether the sample is contaminated (including determining WBC contamination), removal of contaminated fragments, calling sequencing error, etc.

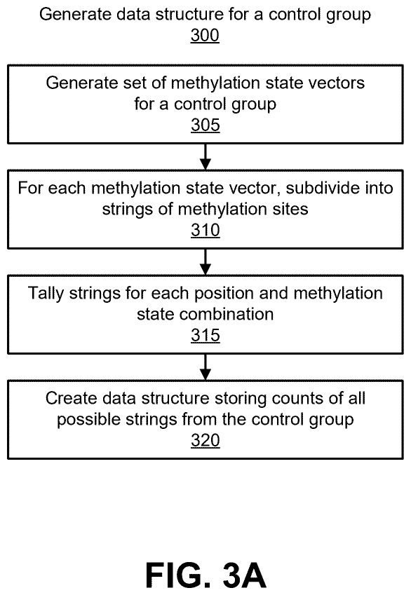

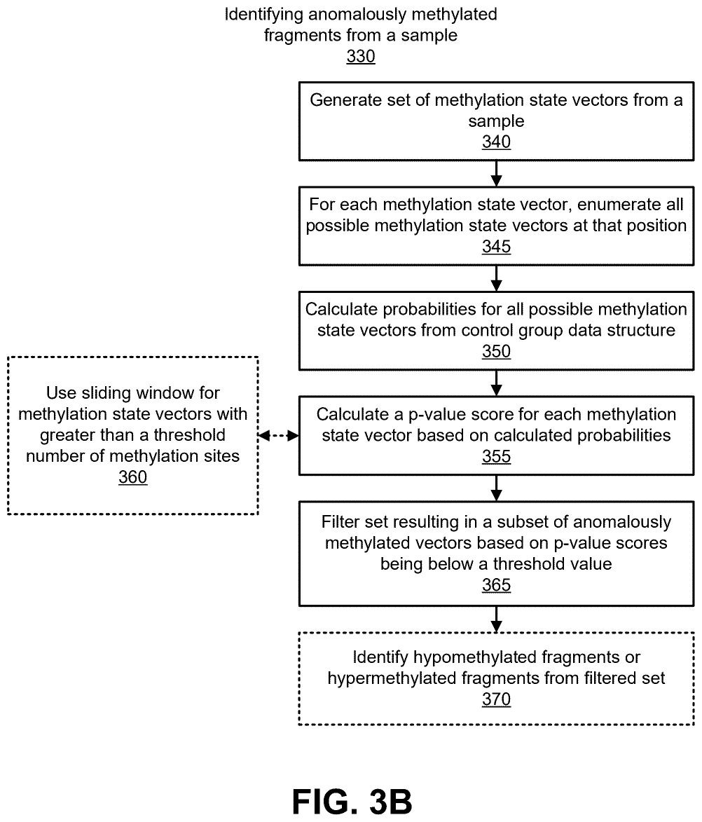

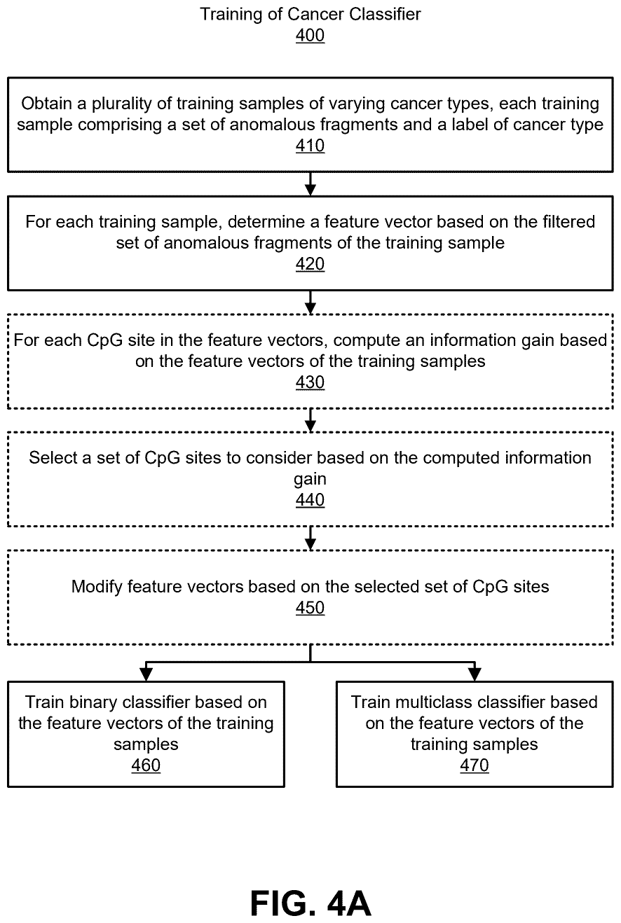

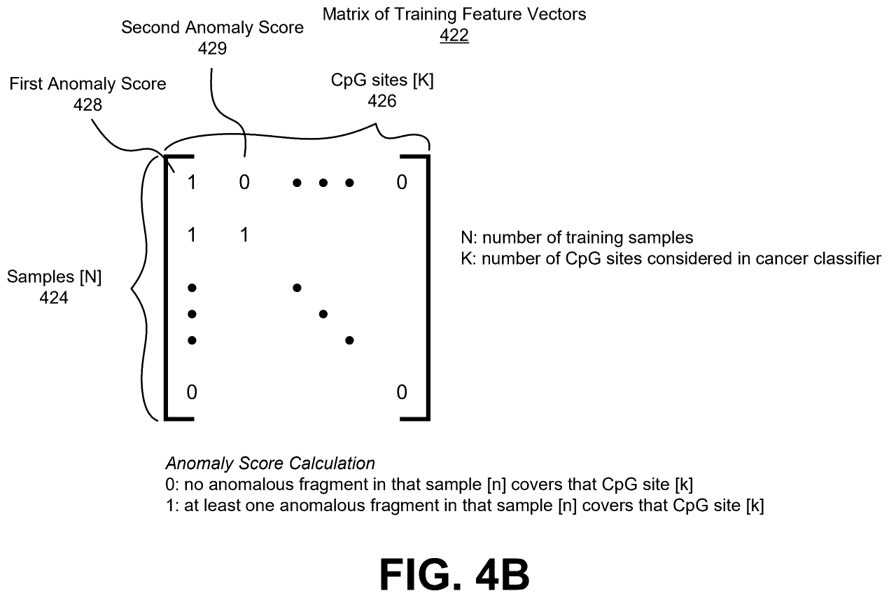

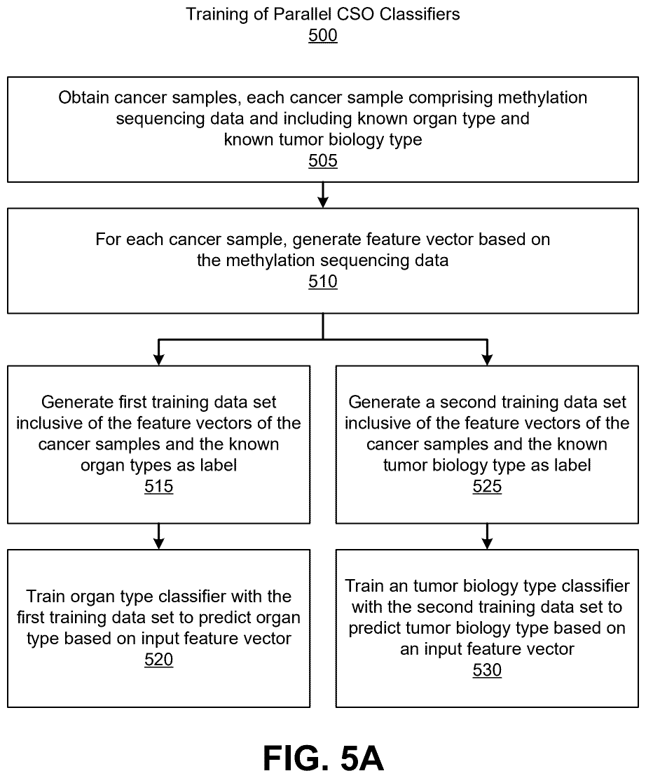

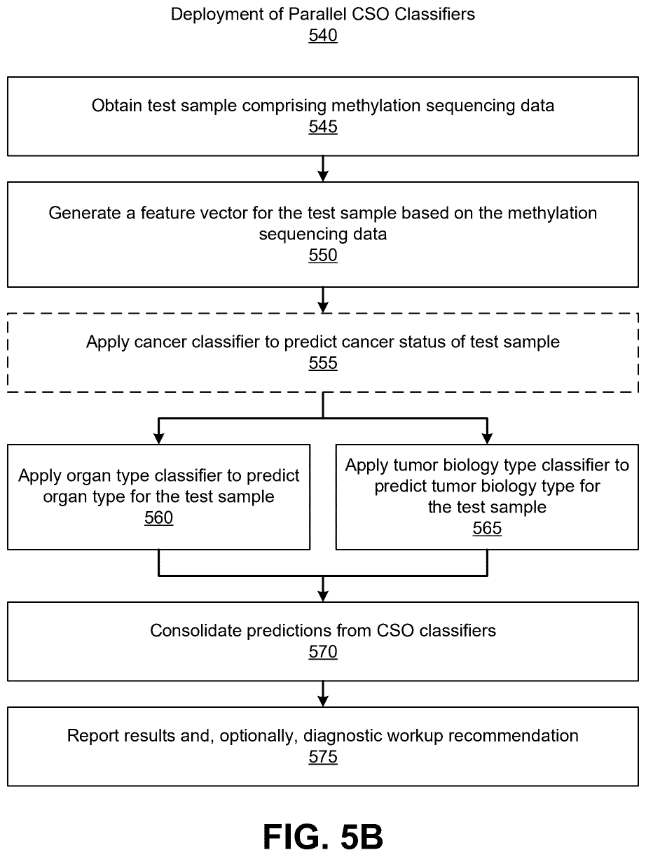

The analytics system performs one or more analyses 140. The analyses are statistical analyses or application of one or more trained models to predict at least a cancer status of the subject from whom the sample is derived. Different genetic features may be evaluated and considered, such as methylation of CpG sites, single nucleotide polymorphisms (SNPs), insertions or deletions (indels), other types of genetic mutation, etc. The analyses 140 may include contamination detection 142, anomalous methylation identification 144 (e.g., further described in FIGS. 3A & 3B), feature extraction 146 (e.g., further described FIGS. 4A, 4B, 5A, and 5B), and cancer classification 148 to determine a cancer prediction (e.g., further described in FIGS. 4A, 4B, 5A, and 5B). Cancer classification generally entails inputting the extracted features to determine a cancer prediction. The cancer prediction may be a label or a value. The label may indicate a particular cancer state. A binary label may indicate presence or absence of a cancer signal; whereas a multiclass label can indicate one or more cancer signal origins from a plurality of potential cancer signal origins that are screened for. Cancer signal origins may be split based on various characteristics of the cancer. For example, cancer signal origins may be split according to organ type and/or tumor biology type. In other examples, cancer signal origins may be further split according to progression (e.g., Stage I, II, III, or IV), prognosis, prediction of response to a candidate treatment, or presence of residual disease after or on treatment. A value may indicate a likelihood of a particular cancer state, e.g., a likelihood of cancer, and/or a likelihood of a particular cancer signal origin.

The analytics system returns the prediction 150 to the healthcare provider. The healthcare provider may establish or adjust a treatment plan based on the cancer prediction. In some embodiments, the analytics system may return to the healthcare provider and/or the subject a diagnostic workup recommendation 160 identifying one or more diagnostic steps that might workup the test result and result in a cancer diagnosis. In such embodiments, the analytics system may store a database associating diagnostic workup options generally accepted by healthcare professionals to be useful diagnostic workup steps in the presence of a predicted cancer signal origin. Optimization of treatment is further described in Section V.D. Treatment. In some embodiments, the analytics system may leverage the cancer classification workflow for prognosis determination, treatment personalization, evaluation of treatment, monitoring cancer status, etc.

### I.B. Methylation Overview

In accordance with the present description, cfDNA fragments from a subject are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy subjects, may provide insight into a subject's cancer status. As is well known in the art, DNA methylation changes (compared to healthy controls) can cause different effects, which may contribute to cancer. Various challenges arise in the identification of informatively methylated cfDNA fragments. First off, determining a DNA fragment to be informatively methylated can hold weight in comparison with a group of control subjects, such that if the control group is small in number, the determination loses confidence due to statistical variability within the smaller size of the control group. Additionally, among a group of control subjects, methylation status can vary which can be difficult to account for when determining a subject's DNA fragments to be informatively methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated. In addition to hypermethylation or hypomethylation, an informative methylation status of a DNA fragment can further be characterized of a sequence of methylated and unmethylated CpGs that are not frequently observed in a healthy population and that is indicative for the presence of cancer or the presence of one or few cancer signal origins.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “cell free nucleic acid” or “cfNA” refers to nucleic acid fragments that circulate in a subject's body (e.g., blood) or are present in other bodily liquids like urine, cerebrovascular fluid, and originate from one or more healthy cells and/or from one or more unhealthy cells (e.g., cancer cells). The term “cell free DNA,” or “cfDNA” refers to deoxyribonucleic acid fragments present in a subject's body outside of the cell (e.g., in blood plasma, urine, sputum, cerebrospinal fluid, etc.).

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of a subject (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “informative fragment,” “anomalously methylated fragment,” or “fragment with an anomalous methylation pattern” refers to a fragment that has informative methylation of CpG sites. Anomalous methylation of a fragment may be determined using probabilistic models to identify unexpectedness of observing a fragment's methylation pattern in a control group.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of 20%, ±10%, +5%, or +1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to ±5%.

As used herein, the term “biological sample,” “patient sample,” or “sample” refers to any sample taken from a subject, which can reflect a biological state associated with the subject, and that includes gDNA or cell-free DNA. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, pericardial fluid, peritoneal fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. A biological sample can include any tissue or material derived from a living or dead subject. A biological sample can be a cell-free sample. A biological sample can comprise a nucleic acid (e.g., DNA or RNA) or a fragment thereof.

The term “nucleic acid” can refer to deoxyribonucleic acid (DNA), ribonucleic acid (RNA) or any hybrid or fragment thereof. The nucleic acid in the sample can be a cell-free nucleic acid. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy subject” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Anomalous cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure, like having a cancer that will respond to a treatment, having residual disease after or during a treatment, or being likely to encounter an event like disease progression, relapse, or even cancer-related death within a pre-specified time frame. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from a subject or multiple subjects. In some embodiments, a reference genome is an assembled or partially assembled genomic sequence from one or more huma subjects. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50×, 100×, etc., where “Y” refers to the number of times a locus is covered with a sequence corresponding to a nucleic acid target, e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

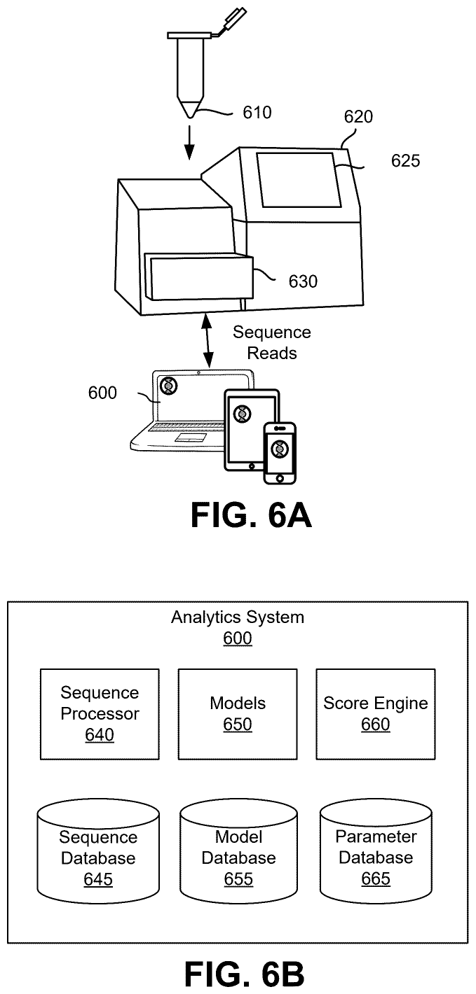

FIG. 6A is an exemplary flowchart of devices for sequencing nucleic acid samples according to one or more embodiments. This illustrative flowchart includes devices such as a sequencer 620 and an analytics system 600. The sequencer 620 and the analytics system 600 may work in tandem to perform one or more steps in the processes.

In various embodiments, the sequencer 620 receives an enriched nucleic acid sample 610. As shown in FIG. 6A, the sequencer 620 can include a graphical user interface 625 that enables user interactions with particular tasks (e.g., initiate sequencing or terminate sequencing) as well as one more loading stations 630 for loading a sequencing cartridge including the enriched fragment samples and/or for loading necessary buffers for performing the sequencing assays. Therefore, once a user of the sequencer 620 has provided the necessary reagents and sequencing cartridge to the loading station 630 of the sequencer 620, the user can initiate sequencing by interacting with the graphical user interface 625 of the sequencer 620. Once initiated, the sequencer 620 performs the sequencing and outputs the sequence reads of the enriched fragments from the nucleic acid sample 610.

In some embodiments, the sequencer 620 is communicatively coupled with the analytics system 600. The analytics system 600 includes some number of computing devices used for processing the sequence reads for various applications such as assessing methylation status at one or more CpG sites, variant calling or quality control. The sequencer 620 may provide the sequence reads in a BAM file format to the analytics system 600. The analytics system 600 can be communicatively coupled to the sequencer 620 through a wireless, wired, or a combination of wireless and wired communication technologies. Generally, the analytics system 600 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 600 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 6B, FIG. 6B is a block diagram of an analytics system 600 for processing DNA samples according to one embodiment. The analytics system implements one or more computing devices for use in analyzing DNA samples. The analytics system 600 includes a sequence processor 640, sequence database 645, model database 655, models 650, parameter database 665, and score engine 660. In some embodiments, the analytics system 600 performs some or all of the processes described throughout this disclosure.

The sequence processor 640 generates methylation state vectors for fragments from a sample. At each CpG site on a fragment, the sequence processor 640 generates a methylation state vector for each fragment specifying a location of the fragment in the reference genome, a number of CpG sites in the fragment, and the methylation state of each CpG site in the fragment whether methylated, unmethylated, or indeterminate via the process 200 of FIG. 2A. The sequence processor 640 may store methylation state vectors for fragments in the sequence database 645. Data in the sequence database 645 may be organized such that the methylation state vectors from a sample are associated to one another.

Further, multiple different models 650 may be stored in the model database 655 or retrieved for use with test samples. In one example, a model is a trained cancer classifier for determining a cancer prediction for a test sample using a feature vector derived from informative fragments. The training and use of the cancer classifier will be further discussed in conjunction with Section III. Cancer Classifier for Determining Cancer. The analytics system 600 may train the one or more models 650 and store various trained parameters in the parameter database 665. The analytics system 600 stores the models 650 along with functions in the model database 655.

During inference, the score engine 660 uses the one or more models 650 to return outputs. The score engine 660 accesses the models 650 in the model database 655 along with trained parameters from the parameter database 665. According to each model, the score engine receives an appropriate input for the model and calculates an output based on the received input, the parameters, and a function of each model relating the input and the output. In some use cases, the score engine 660 further calculates metrics correlating to a confidence in the calculated outputs from the model. In other use cases, the score engine 660 calculates other intermediary values for use in the model.

### II. Sample Sequencing & Processing

### II.A. Generating Methylation State Vectors for DNA Fragments

FIG. 2A is an exemplary flowchart describing a process 200 of sequencing a fragment of cfDNA to obtain a methylation state vector, according to one or more embodiments. In order to analyze DNA methylation, an analytics system first obtains 210 a sample from a subject comprising a plurality of cfDNA molecules. In additional embodiments, the process 200 may be applied to sequence other types of DNA molecules. The process 200 is an embodiment of sample sequencing 120 of FIG. 1.

From the sample, the analytics system can isolate 210 each cfDNA molecule. The cfDNA molecules can be treated 220 to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted cfDNA molecules, a sequencing library can be prepared 230. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 235 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfite) for conversion of unmethylated cytosines to uracils. During enrichment, hybridization probes (also referred to herein as “probes”) can be used to target and pull-down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 240 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

From the sequence reads, the analytics system determines 250 a location and methylation state for each CpG site based on alignment to a reference genome. The analytics system generates 260 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated, whereas an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses.

FIG. 2B is an exemplary illustration of the process 200 of FIG. 2A of sequencing a cfDNA molecule to obtain a methylation state vector, according to one or more embodiments. As an example, the analytics system receives a cfDNA molecule 212 that, in this example, contains three CpG sites. As shown, the first and third CpG sites of the cfDNA molecule 212 are methylated 214. During the treatment step 220, the cfDNA molecule 212 is converted to generate a converted cfDNA molecule 222. During the treatment 220, the second CpG site which was unmethylated has its cytosine converted to uracil. However, the first and third CpG sites were not converted.

After conversion, a sequencing library 230 is prepared and sequenced 240 to generate a sequence read 242. The analytics system aligns 250 the sequence read 242 to a reference genome 244. The reference genome 244 provides the context as to what position in a human genome the fragment cfDNA originates from. In this simplified example, the analytics system aligns 250 the sequence read 242 such that the three CpG sites correlate to CpG sites 23, 24, and 25 (arbitrary reference identifiers used for convenience of description). The analytics system can thus generate information both on methylation status of all CpG sites on the cfDNA molecule 212 and the position in the human genome that the CpG sites map to. As shown, the CpG sites on sequence read 242 which are methylated are read as cytosines. In this example, the cytosines appear in the sequence read 242 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 260 a methylation state vector 252 for the fragment cfDNA 212. In this example, the resulting methylation state vector 252 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight subject lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing. The methylation sequencing assay may comprise probes targeting 500 or more CpG sites, 1,000 or more CpG sites, 1,500 or more CpG sites, 2,000 or more CpG sites, 2,500 or more CpG sites, 3,000 or more CpG sites, 3,500 or more CpG sites, 4,000 or more CpG sites, 4,500 or more CpG sites, 5,000 or more CpG,000 or more CpG sites, sites, 7,000 or more CpG sites, 8,000 or more CpG sites, 9,000 or more CpG sites, 10,000 or more CpG sites, 20,000 or more CpG sites, 30,000 or more CpG sites, 40,000 or more CpG sites, 50,000 or more CpG sites, 60,000 or more CpG sites, 70,000 or more CpG sites, 80,000 or more CpG sites, 90,000 or more CpG sites, 100,000 or more CpG sites, 150,000 or more CpG sites, etc.

For further details regarding methylation sequencing (e.g., WGBS and/or targeted methylation sequencing), see, e.g., U.S. patent application Ser. No. 16/719,902, entitled “Systems and Methods for Estimating Cell Source Fractions Using Methylation Information,” filed Dec. 18, 2019, each of which is hereby incorporated by reference. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments can comprise 1,000 or more nucleic acid methylation fragments, 5,000 or more nucleic acid methylation fragments, 10,000 or more nucleic acid methylation fragments, 20,000 or more nucleic acid methylation fragments, 30,000 or more nucleic acid methylation fragments, 40,000 or more nucleic acid methylation fragments, or 50,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 480 nucleotides. An average length of a corresponding plurality of nucleic acid methylation fragments can be between 100 and 600 nucleotides. An average length of a corresponding plurality of nucleic acid methylation fragments can be between 50 and 750 nucleotides. An average length of a corresponding plurality of nucleic acid methylation fragments can be between 30 and 1,000 nucleotides.

Further details regarding methods for sequencing nucleic acids and methylation sequencing data are disclosed in U.S. patent application Ser. No. 17/191,914, titled “Systems and Methods for Cancer Condition Determination Using Autoencoders,” filed Mar. 4, 2021, which is hereby incorporated herein by reference in its entirety.

### III. Cancer Classification for Determining Cancer

Cancer classification involves extracting genetic features and applying one or more models to the extracted features to determine a cancer prediction. The analytics system aggregates extracted features into a feature vector which can then be input into a trained cancer prediction model to determine a cancer prediction based on the input feature vector. The cancer prediction may comprise one or more labels and/or one or more values. One label may be binary, indicating a presence or absence of cancer in the test subject. Another label may be multiclass, indicating one or more particular cancer signal origins from a plurality of screened cancer signal origins. One value may indicate a likelihood of presence of cancer. Another value may indicate a likelihood of absence of cancer. Yet another value may otherwise indicate another prognosis of the cancer. For example, the value may quantify a progression and/or potential response to treatment of the cancer.

In one or more embodiments, the feature vectors input into the cancer classifier are based on a set of informative fragments (also referred to as “anomalously methylated” or “unusual fragments of extreme methylation” (UFXM)) determined from the test sample.

In some embodiments, a cancer classifier may be a machine-learned model which is a computation model comprising a plurality of classification parameters and a function representing a relation between the feature vector as input and the cancer prediction as output. Inputting the feature vector into the function with the classification parameters yields the cancer prediction. The machine-learned model may be trained using training samples derived from subjects with known cancer diagnoses. The training samples may be divided into cohorts of varying labels. For example, there may be a cohort of training samples for each cancer signal origin.

A machine-learned model may be trained through any combination of machine-learning techniques including, but not limited to, Linear Regression, Logistic Regression, Support Vector Machines (SVM), K-Nearest Neighbors (KNN), Decision Trees (e.g., ID3, C4.5, CART), Random Forest, Neural Networks (e.g., Multi-Layer Perceptron, Convolutional Neural Networks, Recurrent Neural Networks), Naive Bayes Classifier, Gradient Boosting Machines (e.g., XGBoost, LightGBM, CatBoost), AdaBoost, K-Means Clustering, Hierarchical Clustering, DBSCAN (Density-Based Spatial Clustering of Applications with Noise), Gaussian Mixture Models (GMM), PCA (Principal Component Analysis), t-SNE (t-Distributed Stochastic Neighbor Embedding), UMAP (Uniform Manifold Approximation and Projection), ICA (Independent Component Analysis), Autoencoders (e.g., Variational Autoencoders, Denoising Autoencoders), Self-Organizing Maps (SOM), Q-Learning, Deep Q Network (DQN), SARSA (State-Action-Reward-State-Action), Monte Carlo Methods, Temporal Difference Learning (TD Learning), Policy Gradients (e.g., REINFORCE, Actor-Critic), Proximal Policy Optimization (PPO), Advantage Actor-Critic (A2C), Soft Actor-Critic (SAC), Deep Deterministic Policy Gradient (DDPG), Hidden Markov Models (HMM), Conditional Random Fields (CRF), Latent Dirichlet Allocation (LDA), Restricted Boltzmann Machines (RBM), Genetic Algorithms, and Swarm Intelligence Algorithms (e.g., Particle Swarm Optimization, Ant Colony Optimization), Expectation Maximization (EM).

### III.A. Identifying Informative Fragments

The analytics system can determine informative fragments for a sample using the sample's methylation state vectors. For each fragment in a sample, the analytics system can determine whether the fragment is an informative fragment using the methylation state vector corresponding to the fragment. In some embodiments, the analytics system calculates a p-value score for each methylation state vector describing a probability of observing that methylation state vector or other methylation state vectors even less probable in the healthy control group. The process for calculating a p-value score is further discussed below in Section III.A.i. P-Value Filtering. The analytics system may determine fragments with a methylation state vector having below a threshold p-value score as informative fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining informative fragments. Examples of other probabilistic models include a mixture model, a deep probabilistic model, etc. In some embodiments, the analytics system may use any combination of the processes described below for identifying informative fragments. With the identified informative fragments, the analytics system may filter the set of methylation state vectors for a sample for use in other processes, e.g., for use in training and deploying a cancer classifier.

### III.A.I. P-Value Filtering

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be anomalously methylated, the analytics system can use a healthy control group with a majority of fragments that are normally methylated. When conducting this probabilistic analysis for determining informative fragments, the determination can hold weight in comparison with the group of control subjects that make up the healthy control group. To bolster robustness in the healthy control group, the analytics system may select some threshold number of healthy subjects to source samples including DNA fragments. FIG. 3A below describes the method of generating a data structure for a healthy control group with which the analytics system may calculate p-value scores. FIG. 3B describes the method of calculating a p-value score with the generated data structure.

FIG. 3A is a flowchart describing a process 300 of generating a data structure for a healthy control group, according to an embodiment. To create a healthy control group data structure, the analytics system can receive a plurality of DNA fragments (e.g., cfDNA) from a plurality of healthy subjects. The analytics system can generate 305 a methylation state vector for each fragment, for example via the process 200 of FIG. 2A.

With each fragment's methylation state vector, the analytics system can subdivide 310 the methylation state vector into strings of CpG sites. In some embodiments, the analytics system subdivides 310 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 can result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 315 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 310 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site in the reference genome. The analytics system creates 315 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is anomalous or not.

FIG. 3B is a flowchart describing a process 330 for identifying anomalously methylated fragments from a subject, according to an embodiment. In process 330, the analytics system generates 340 methylation state vectors from cfDNA fragments of the subject, e.g., via the process 200 of FIG. 2A. The analytics system can handle each methylation state vector as follows.

For a given methylation state vector, the analytics system enumerates 345 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2ⁿ possibilities of methylation state vectors. With methylation state vectors inclusive of indeterminate states for one or more CpG sites, the analytics system may enumerate 330 possibilities of methylation state vectors considering only CpG sites that have observed states.

The analytics system calculates 350 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMMs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 355 a p-value score for the methylation state vector using the calculated probabilities for each possibility. In some embodiments, this includes identifying the calculated probability corresponding to the possibility that matches the methylation state vector in question. Specifically, this can be the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy subject, and which causes the fragment to be labeled anomalously methylated, relative to the healthy control group. A high p-value score can generally relate to a methylation state vector that is expected to be present, in a relative sense, in a healthy subject. If the healthy control group is a non-cancerous group, for example, a low p-value can indicate that the fragment is anomalous methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are anomalously methylated, the analytics system may filter 365 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 300, the analytics system can yield a median (range) of 2,800 (1,500-12,000) fragments with anomalous methylation patterns for participants without cancer in training, and a median (range) of 3,000 (1,200-420,000) fragments with anomalous methylation patterns for participants with cancer in training. These filtered sets of fragments with anomalous methylation patterns may be used for the downstream analyses as described below in Sections III.B & III.C.

In some embodiments, the analytics system uses 360 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytics system can calculate a p-value score for the window including the first CpG site. The analytics system can then “slide” the window to the second CpG site in the vector, and calculates another p-value score for the second window. Thus, for a window size l and methylation vector length m, each methylation state vector can generate m−l+l p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2 \hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of informative fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytics system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of 1's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. patent application Ser. No. 17/191,914, titled “Systems and Methods for Cancer Condition Determination Using Autoencoders,” filed Mar. 4, 2021, which is hereby incorporated herein by reference in its entirety.

The respective nucleic acid methylation fragment may fail to satisfy a selection criterion in the one or more selection criteria when the respective nucleic acid methylation fragment has an anomalous methylation score that is less than an anomalous methylation score threshold. In this situation, the anomalous methylation score can be determined by a mixture model. For example, a mixture model can detect an anomalous methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An anomalous methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 4, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 4, 5, or more than 5. For example, a first nucleic acid methylation fragment having the same corresponding genomic start and end position as a second nucleic acid methylation fragment but having at least 1, at least 2, at least 3, at least 4, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

### III.A.II. Hypermethylated Fragments and Hypomethylated Fragments

In some embodiments, the analytics system 370 determines hypomethylated fragments or hypermethylated fragments from the filtered set as informative fragments. The analytics system identifies hypermethylated fragments having over a threshold number of CpG sites and over a threshold percentage of the CpG sites methylated. The analytics system identifies hypomethylated fragments having over the threshold number of CpG sites and over a threshold percentage of CpG sites unmethylated. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### III.B. Training of Cancer Classifier

FIG. 4A is a flowchart describing a process 400 of training a cancer classifier, according to an embodiment. The analytics system obtains 410 a plurality of training samples each having a set of informative fragments and a label of a cancer signal origin. The plurality of training samples can include any combination of samples from healthy subjects with a general label of “non-cancer,” samples from subjects with a general label of “cancer” or a specific label (e.g., “breast cancer,” “lung cancer,” etc.). The training samples from subjects for one cancer signal origin may be termed a cohort for that cancer signal origin or a cancer signal origin cohort.

The analytics system determines 420, for each training sample, a feature vector based on the set of informative fragments of the training sample. The analytics system can calculate an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an informative fragment in the set of informative fragments that encompasses the CpG site. In another embodiment, the analytics system defines the anomaly score based on a count of informative fragments overlapping the CpG site. In one example, the analytics system may use a trinary scoring assigning a first score for lack of presence of informative fragments, a second score for presence of a few informative fragments, and a third score for presence of more than a few informative fragments. For example, the analytics system counts 5 informative fragment in a sample that overlap the CpG site and calculates an anomaly score based on the count of 5.

Once all anomaly scores are determined for a training sample, the analytics system can determine the feature vector as a vector of elements including, for each element, one of the anomaly scores associated with one of the CpG sites in an initial set. The analytics system can normalize the anomaly scores of the feature vector based on a coverage of the sample. Here, coverage can refer to a median or average sequencing depth over all CpG sites covered by the initial set of CpG sites used in the classifier, or based on the set of informative fragments for a given training sample.

As an example, reference is now made to FIG. 4B illustrating a matrix of training feature vectors 422. In this example, the analytics system has identified CpG sites [K]426 for consideration in generating feature vectors for the cancer classifier. The analytics system selects training samples [N]424. The analytics system determines a first anomaly score 428 for a first arbitrary CpG site [k1] to be used in the feature vector for a training sample [n1]. The analytics system checks each informative fragment in the set of informative fragments. If the analytics system identifies at least one informative fragment that includes the first CpG site, then the analytics system determines the first anomaly score 428 for the first CpG site as 1, as illustrated in FIG. 4B. Considering a second arbitrary CpG site [k2], the analytics system similarly checks the set of informative fragments for at least one that includes the second CpG site [k2]. If the analytics system does not find any such informative fragment that includes the second CpG site, the analytics system determines a second anomaly score 429 for the second CpG site [k2] to be 0, as illustrated in FIG. 4B. Once the analytics system determines all the anomaly scores for the initial set of CpG sites, the analytics system determines the feature vector for the first training sample [n1] including the anomaly scores with the feature vector including the first anomaly score 428 of 1 for the first CpG site [k1] and the second anomaly score 429 of 0 for the second CpG site [k2] and subsequent anomaly scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety. The various featurization approaches may generate distinct features to be included in a sample's feature vector.

In some embodiments, each classifier may be trained on different matrices of training feature vectors. For example, one classifier may be trained on a first matrix covering a first set of genomic regions, whereas another classifier may be trained on a second matrix covering a second, differing set of genomic regions. In another example, one classifier may be trained on a first matrix with features determined according to one featurization approach, whereas another classifier may be trained on a second matrix with features determined according to another featurization approach.

The analytics system may further limit the CpG sites considered for use in the cancer classifier. The analytics system computes 430, for each CpG site in the initial set of CpG sites, an information gain based on the feature vectors of the training samples. From step 420, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer signal origins, or may be duplicative with other CpG sites.

In one embodiment, the analytics system computes 430 an information gain for each cancer signal origin and for each CpG site in the initial set to determine whether to include that CpG site in the classifier. The information gain is computed for training samples with a given cancer signal origin compared to all other samples. For example, two random variables ‘informative fragment’ (‘IF’) and ‘cancer signal’ (‘CS’) are used. In one embodiment, IF is a binary variable indicating whether there is an informative fragment overlapping a given CpG site in a given samples as determined for the anomaly score/feature vector above. CS is a random variable indicating whether the cancer signal has a particular origin, for example a particular organ or organ group, or a particular cancer biology. The analytics system computes the mutual information with respect to CS given IF. That is, how many bits of information about the cancer signal origin are gained if it is known whether there is an informative fragment overlapping a particular CpG site. In practice, for a first cancer signal origin, the analytics system computes pairwise mutual information gain against each other cancer signal origin and sums the mutual information gain across all the other cancer signal origins.

For a given cancer signal origin, the analytics system can use this information to rank CpG sites based on how cancer specific they are. This procedure can be repeated for all cancer signal origins under consideration. If a particular region is commonly anomalously methylated in training samples of a given cancer but not in training samples of other cancer signal origins or in healthy training samples, then CpG sites overlapped by those informative fragments can have high information gains for the given cancer signal origin. The ranked CpG sites for each cancer signal origin can be greedily added (selected) 440 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 450 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 420 or to the selected set of CpG sites from step 450. In one embodiment, the analytics system trains 460 a binary cancer classifier to distinguish between cancer and non-cancer based on the feature vectors of the training samples. In this manner, the analytics system uses training samples that include both non-cancer samples from healthy subjects and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 470 a multiclass cancer classifier to distinguish between many cancer signal origins (also referred to as CSO labels). CSO can include one or more organs, organ groups, classes for cancer biology, cellular lineage of a cancer cell, or cancer drivers like a viral status. To do so, the analytics system can use the cancer signal origin cohorts and may also include or not include a non-cancer cohort. In this multiclass embodiment, the cancer classifier is trained to determine a cancer prediction (or, more specifically, a CSO prediction) that comprises a prediction value for each of the cancer signal origins being classified for. The prediction values may correspond to a likelihood that a given training sample (and during inference, a test sample) has each of the cancer signal origins. In one implementation, the prediction values are scored between 0 and 100, wherein the cumulation of the prediction values equals 100. For example, the cancer classifier returns a cancer prediction including a prediction value for the affected organ or organ group being breast, lung, and non-cancer. For example, the classifier can return a cancer prediction that a test samples signal origin is 65% likelihood of breast, 25% likelihood of lung, and 10% likelihood of non-cancer. The analytics system may further evaluate the prediction values to generate a prediction of a presence of one or more cancers in the sample, also may be referred to as a CSO prediction indicating one or more CSO labels, e.g., a first CSO prediction with the highest prediction value, a second CSO prediction with the second highest prediction value, etc. Continuing with the example above and given the percentages, in this example the system may determine that the sample's cancer signal origin is breast given that the CSO breast has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc. In some embodiments, such models may include 1,000 or more parameters, 2,000 or more parameters, 3,000 or more parameters, 4,000 or more parameters, 5,000 or more parameters, 10,000 or more parameters, 15,000 or more parameters, 20,000 or more parameters, 25,000 or more parameters, 50,000 or more parameters, 100,000 or more parameters, 150,000 or more parameters, 200,000 or more parameters, 250,000 or more parameters, 300,000 or more parameters, 350,000 or more parameters, 400,000 or more parameters, 450,000 or more parameters, or 500,000 or more parameters.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

### III.C. Deployment of Cancer Classifier

During use of the cancer classifier, the analytics system can obtain a test sample from a subject of unknown cancer signal origin. The analytics system may process the test sample comprised of DNA molecules with any combination of the processes 200 and 330 to achieve a set of informative fragments. The analytics system can determine a test feature vector for use by the cancer classifier according to similar principles discussed in the process 400. The analytics system can calculate an anomaly score for each CpG site in a plurality of CpG sites in use by the cancer classifier. For example, the cancer classifier receives as input feature vectors inclusive of anomaly scores for 1,000 selected CpG sites. The analytics system can thus determine a test feature vector inclusive of anomaly scores for the 1,000 selected CpG sites based on the set of informative fragments. The analytics system can calculate the anomaly scores in a same manner as the training samples. In some embodiments, the analytics system defines the anomaly score as a binary score based on whether there is a hypermethylated or hypomethylated fragment in the set of informative fragments that encompasses the CpG site.

The analytics system can then input the test feature vector into the cancer classifier. The function of the cancer classifier can then generate a cancer prediction based on the classification parameters trained in the process 400 and the test feature vector. In the first manner, the cancer prediction can be binary and selected from a group consisting of “cancer” or non-cancer;” in the second manner, the cancer prediction is selected from a group of many potential cancer signal origins that could identify affected organ or organ group and at the same time the underlying cancer biology like a histologic type, the cellular lineage of the cancer cell or origin, or the presence of a cancer driver like infection with an oncovirus and “non-cancer.” In additional embodiments, the cancer prediction has prediction values for each of the many cancer signal origins. Moreover, the analytics system may determine that the test sample's cancer signal has most likely one of the cancer signal origins. Following the example above with the cancer prediction for a test sample as 65% likelihood of originating from the organ breast, 25% likelihood of originating from the organ lung, and 10% likelihood of non-cancer, the analytics system may determine that the test sample is most likely to have a cancer of origin breast. In another example, where the cancer prediction is binary as 60% likelihood of non-cancer and 40% likelihood of cancer, the analytics system determines that the test sample is most likely to not have a cancer signal. In additional embodiments, the cancer prediction with the highest likelihood may still be compared against a threshold (e.g., 40%, 50%, 60%, 70%) in order to call the test subject as having a cancer signal or that cancer signal origin present. If the cancer prediction with the highest likelihood does not surpass that threshold, the analytics system may return an inconclusive result, or report the presence of a cancer signal, while not reporting a cancer signal origin

In additional embodiments, the analytics system chains a cancer classifier trained in step 460 of the process 400 with one or more additional cancer classifiers trained in step 470 of the process 400. The analytics system can input the test feature vector into the cancer classifier trained as a binary classifier in step 460 of the process 400. The analytics system can receive an output of a cancer prediction. The cancer prediction may be binary as to whether the test subject likely has or likely does not have cancer and if a cancer signal is detected in the sample. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer signal origins. The multiclass cancer classifier can receive the test feature vector and returns one or more cancer signal predictions of a cancer signal origin of the plurality of cancer signals in one or multiple categories of cancer signal origins. For example, the multiclass cancer classifier provides a cancer prediction specifying that the test subject is most likely to have a cancer signal originating in a predicted organ or organ group like ovaries or Fallopian tube cancer while at the same time providing a cancer signal origin prediction that this cancer signal has a cell of Mullerian lineage as origin. In another implementation, the multiclass cancer classifier provides a prediction value for each cancer signal origin of the plurality of cancer signal origins and cancer signal origin classifiers. For example, a cancer prediction may include a cancer signal origin for head and neck of 40%, a cancer signal origin for anus of 20%, and cancer signal origin of cervix of 20% for the organ of organ group, and in parallel a prediction of a cancer signal origin being 80% a carcinoma associated with a Human Papilloma Virus (HPV) infection, 10% having origin in a squamous cell carcinoma, and 10% having origin in a cancer of cells of Mullerian lineage.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using CSO thresholding based on one or more CSO predictions. The analytics system may further generate a feature vector for the test sample for use in the multiclass cancer classifier to determine a cancer signal origin prediction indicating one or more likely organ or organ groups as cancer signal origins, and in parallel indicating one or more histologic types, cellular lineages, or oncogenic drives as cancer signal origin.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### IV. Parallel Cancer Source of Origin Classifiers

The analytics system may implement cancer signal origin classifiers to predict cancer signal origin characteristics. The cancer signal origin classifiers may be embodiments of the cancer classifier trained according to FIG. 4A. Cancers can be characterized according to their cancer signal origin characteristics. For example, cancer signal origin may include the organ or organ group that is primarily affected by the cancer and/or a tumor biology type that represents how the cancer behaves, or what its originating cellular lineage or oncogenic drivers are. Cancer signal origin may further be parsed based on cell type that is primarily affected. Each of the types (organ or organ group, cell type, and tumor biology type) may be independent variables and obtain independent predictions, e.g., one organ or organ group can be crossed with one of many tumor biology signal origins, etc.

The results of the cancer source of origin classifiers may be used to inform workup steps to work up a detected cancer signal origin to obtain the diagnosis of a cancer or a confident assessment that the cancer signal detection was false positive and that a patient is not afflicted with a cancer. In some embodiments, the results of the cancer signal origin classifiers are provided to a healthcare provider to inform options for a diagnostic workup of a detected cancer signal. In other embodiments, the analytics system may identify one or more diagnostic workup options to recommend to a healthcare provider based on the results of the cancer signal origin classifiers. The results of the cancer signal origin classifiers may provide added detail to the cancer prediction that permits the diagnostic workup to be better tailored to the subject's cancer.

### IV.A. Organ Types & Tumor Biology Types

In one or more embodiments, the organ types and tumor biology types may be orthogonal. For example, any organ or organ-group signal origin can be paired with many tumor biology signal origins. In other embodiments, additional cancer signal origin characteristics may be used (e.g., cell type).

In one or more embodiments, the organ or organ group signal origin may include: breast; prostate; lung; head or neck; anus; cervix; ovary or fallopian tubes; uterus; bladder or urothelial; kidney; stomach or esophagus; liver or intrahepatic bile duct; pancreas, extrahepatic bile duct, or gall bladder; colon or rectum; bone or soft tissue; skin; blood, lymphatic system, or bone marrow; thyroid; ambiguous or absent cancer signal origin signal; or some combination thereof. In one or more embodiments, some organ types may be further subdivided, e.g., the stomach or esophagus signal origin can be split into a signal origin stomach and a signal origin esophagus.

In one or more embodiments, cancers with signal origin breast includes cancers such as invasive ductal breast carcinoma, breast cancer of no special type (NST), invasive lobular breast carcinoma, or some combination thereof. Example cancers that may have a different cancer signal origin include sarcoma (with the signal origin bone or soft tissue) or lymphoma (with signal origin blood, lymphatic system, or bone marrow) reported in the breast, any invasive skin cancer of the breast (with signal origin skin), Paget's disease, or some combination thereof.

In one or more embodiments, the cancer signal origin prostate includes cancers such as invasive ductal prostate adenocarcinoma, invasive acinar adenocarcinoma of the prostate, small cell carcinomas of the prostate, or some combination thereof. An example cancer that may have a different cancer signal origin includes sarcoma (with cancer signal origin bone or soft tissue) or lymphoma (with cancer signal origin bone, lymphatic system, or bone marrow) reported in the prostate.

In one or more embodiments, the cancer signal origin lung includes cancers such as lung adenocarcinoma, lung squamous cell carcinoma, non-small-cell lung cancer not-otherwise specified (NSCLC NOS), small-cell lung cancer (SCLC), carcinoid in the lung, or some combination thereof. An example cancer that may have a different cancer signal origin includes sarcoma or lymphoma reported in the lung, or some combination thereof.

In one or more embodiments, the cancer signal origin head or neck includes cancers such as oropharyngeal human-papillomavirus-associated (HPV-associated) squamous cell carcinoma, HPV-negative squamous cell carcinoma of the larynx, salivary gland adenocarcinoma, or some combination thereof. Example cancers that may have a different cancer signal origin include sarcoma or lymphoma reported in the head & neck region, skin cancers reported in the head & neck region, or some combination thereof.

In one or more embodiments, the cancer signal origin anus may include cancers such as HPV-associated squamous cell carcinoma of the anus, anal gland adenocarcinoma, HPV-positive squamous cell carcinoma reported with primary site rectum, or some combination thereof. Example cancers that may have a different cancer signal origin include skin cancers reported in the anal region, extramammary Paget disease in the region, squamous cell carcinoma reported in the colon (even if HPV-positive), sarcoma or lymphoma reported in the anal region, or some combination thereof.

In one or more embodiments, the cancer signal origin cervix may include cancers such as HPV-associated squamous cell carcinoma of the cervix, HPV-associated adenocarcinoma of the cervix, neuroendocrine carcinoma of the cervix, non-HPV-associated adenocarcinoma of the cervix, or some combination thereof.

In one or more embodiments, the cancer signal origin ovary or fallopian tubes may include cancers such as fallopian-tube-derived serous cystadenocarcinoma of the ovaries, endometrioid ovarian carcinoma, malignant mullerian mixed carcinoma of the ovaries, clear cell carcinoma of the ovaries, small cell carcinoma of the ovaries, malignant Brenner tumor, or some combination thereof. Example cancers that may have a different cancer signal origin include germ cell tumors, sarcoma or lymphoma reported in the ovaries or the peritoneal region, mesothelioma in the peritoneal region, or some combination thereof.

In one or more embodiments, the cancer signal origin uterus may include cancers such as endometrial carcinoma, carcinosarcoma of the uterus, high-grade serous cystadenocarcinoma reported with uterus as primary site, or some combination thereof. Example cancers that may have a different cancer signal origin include germ cell tumors, uterine sarcomas, endometrial stromal tumors, or some combination thereof.

In one or more embodiments, the cancer signal origin bladder or urothelial may include cancers such as adenocarcinoma of the bladder, transitional cell carcinoma of the bladder, urothelial carcinomas of the ureter or renal pelvis, transitional or renal cell carcinomas reported in the kidney, urothelial carcinoma of the renal pelvis, carcinoma of the ureter, small cell carcinoma of the bladder, adenocarcinoma NOS in the renal pelvis, or some combination thereof. Example cancers that may have a different cancer signal origin include sarcoma or lymphoma reported in the bladder, ureter, or renal pelvis, urothelial carcinoma in the kidney, or some combination thereof.

In one or more embodiments, the cancer signal origin kidney includes Renal cell carcinoma, carcinoid in kidney, adenocarcinoma NOS in the kidney, or some combination thereof. Example cancers that may have a different cancer signal origin include sarcoma or lymphoma reported in the kidney, transitional or urothelial carcinomas reported in the kidney, or some combination thereof.

In one or more embodiments, the cancer signal origin stomach or esophagus may include gastric adenocarcinoma, esophageal adenocarcinoma, esophageal squamous cell carcinoma, small cell carcinoma in stomach, carcinoid in stomach, or some combination thereof. Example cancers that may have a different cancer signal origin include gastrointestinal stromal tumor (GIST), mucosa associated lymphoid tissue (MALT) lymphoma in stomach, cancers of the small intestines, or some combination thereof.

In one or more embodiments, the cancer signal origin liver or intrahepatic bile duct includes cancers such as hepatocellular carcinoma, intrahepatic cholangiocarcinoma, small cell carcinoma in the liver, carcinoid in the liver, or some combination thereof. Example cancers that may have a different cancer signal origin include carcinoma of the perihilar bile duct, sarcoma or lymphoma reported in the liver, or some combination thereof.

In one or more embodiments, the cancer signal origin pancreas, extrahepatic bile ducts, or gallbladder includes cancers such as pancreatic ductal adenocarcinoma, gallbladder adenocarcinoma, extrahepatic bile duct carcinoma, cystic duct cholangiocarcinoma, pancreatic neuroendocrine tumor, or some combination thereof. Example cancers that may have a different cancer signal origin include sarcoma or lymphoma reported in pancreas, gallbladder, or bile ducts, intrahepatic cholangiocarcinoma, or some combination thereof.

In one or more embodiments, the cancer signal origin colon or rectum may include cancers such as colorectal adenocarcinoma, signet ring cell carcinoma of colon, large cell neuroendocrine carcinoma in colon, carcinoid in colon, adenocarcinoma in the appendix, carcinoid of the appendix, squamous cell carcinoma in colon, or some combination thereof. Example cancers that may have a different cancer signal origin include HPV-positive squamous cell carcinoma reported in the rectum, sarcoma or lymphoma reported in colon or rectum, adenocarcinoma or carcinoid of the small intestines, or some combination thereof.

In one or more embodiments, the cancer signal origin bone or soft tissue may include cancers such as uterine leiomyosarcoma, malignant solitary fibrous tumor, osteosarcoma, gastrointestinal stromal tumor, malignant fibrous histiocytoma (of skin), malignant Hemangiopericytoma in the brain, or some combination thereof. An example cancer that may have a different cancer signal origin includes myeloid sarcoma.

In one or more embodiments, the cancer signal origin skin may include cancers such as melanoma of extremities, melanoma in the head & neck region, Merkel cell carcinoma, digital papillary adenocarcinoma, or some combination thereof. Example cancers that may have a different cancer signal origin include basal cell carcinoma of skin (unless metastatic), squamous cell carcinoma of skin (unless metastatic), cutaneous lymphomas, or some combination thereof.

In one or more embodiments, the cancer signal origin blood, lymphatic system, or bone marrow includes cancers such as lymphomas, including CNS lymphomas and MALT lymphomas of the GI tract, lymphoid leukemia, myeloid leukemia, multiple myeloma or plasma cell myeloma, or some combination thereof. An example cancer that may have a different cancer signal origin includes hematologic pre-cursor conditions.

In one or more embodiments, the cancer signal origin thyroid may include cancers such as medullary carcinoma of the thyroid, papillary carcinoma of the thyroid, or some combination thereof. An example cancer that may have a different cancer signal origin includes sarcoma or lymphoma reported in the thyroid.

In one or more embodiments, there might be no cancer signal or an ambiguous cancer signal origin assigned to cancers such as mesothelioma, cancer of small intestines, cancer of penis, vulva, or vagina, cancers of clinically unknown primary, multiple primaries, cancers of brain and spinal cord (other than sarcomas and lymphomas), or some combination thereof.

In one or more embodiments, the cancer signal origin classes for tumor biology include classes identifying the cell lineage of the tumor cell or origin, a histologic type of a tumor, or an oncogenic driver. Cancer signal origin identifying cell lineage include lymphoid neoplasm, myeloid neoplasm, plasma cell neoplasm, neuroendocrine carcinoma or tumor, neoplasm of Mullerian origin, mesenchymal tumor, melanocytic neoplasm, mesothelial neoplasm. Cancer signal origin identifying a histologic type includes adenocarcinoma, squamous cell carcinoma (not HPV-associated), hepatocellular carcinoma, transitional cell carcinoma. Cancer signal origin identifying an oncogenic driver include HPV-associated carcinoma. There can further be a cancer signal origin identifying any other tumor biology, while some cases may have no or an ambiguous tumor biology, or some combination thereof assigned.

In one or more embodiments, the cancer signal origin lymphoid neoplasm includes cancers such as Hodgkin and non-Hodgkin lymphoma, T-cell lymphoma, B-lymphoblastic lymphoma (BLL), small lymphocytic lymphoma (SLL), primary cutaneous follicle center lymphoma, precursor B and T lymphoblastic leukemia, gastric mucosa-associated lymphoid tissue lymphoma, or some combination thereof. Example cancers that may be excluded include premalignant hematologic conditions. While plasma cells differentiate from B-lymphocytes, all malignancies with plasma cell origin have the cancer signal origin plasma cell neoplasms instead.

In one or more embodiments, the cancer signal origin myeloid neoplasm includes cancers such as acute myeloid leukemia (AML), chronic myeloid leukemia (CML), myelodysplastic syndromes (MDS), malignant mastocytosis, myeloid sarcoma, acute erythroid leukemia, or some combination thereof. An example cancer that may have a different cancer signal origin in this cancer signal origin includes premalignant hematologic conditions.

In one or more embodiments, the cancer signal origin plasma cell neoplasm includes multiple myeloma, plasma cell myeloma, or some combination thereof. An example cancer that may be excluded includes premalignant hematologic conditions.

In one or more embodiments, the cancer signal origin neuroendocrine carcinoma includes cancers such as SCLC, small cell carcinoma in the prostate, large cell neuroendocrine carcinoma in the colon, Medullary carcinoma of the thyroid, typical and atypical carcinoids (functional or non-functional), neuroendocrine carcinomas NOS, pancreatic neuroendocrine tumors, collision tumor of SCLC and adenocarcinoma in the lung or combined small cell carcinomas, adenocarcinoma with neuroendocrine differentiation, mixed neuroendocrine non-neuroendocrine neoplasms with low-grade neuroendocrine component, or some combination thereof.

In one or more embodiments, the cancer signal origin adenocarcinoma includes cancers such as colorectal adenocarcinoma, bronchoalveolar or minimally invasive adenocarcinoma of the lung, bladder adenocarcinoma if not reported as transitional cell carcinoma, signet ring cell carcinoma of the stomach, pancreatic ductal adenocarcinoma, acinar prostate carcinoma, intrahepatic or extrahepatic bile duct cholangiocarcinoma, follicular carcinoma of the thyroid, adenocarcinoma of the salivary glands, or some combination thereof. Example cancers that may have a different cancer signal origin include HPV-positive adenocarcinomas (their cancer signal origin is HPV-associated carcinomas), transitional cell carcinomas of the bladder, adenocarcinomas from a cell of Mullerian duct embryologic origin. Mixed or collision tumors between an adenocarcinoma and a high-grade neuroendocrine carcinoma have the cancer signal origin neuroendocrine tumor or carcinoma, Subtypes or adenocarcinomas of lineages that are included in other class specifications have the cancer signal origin representing that lineage.

In one or more embodiments, the cancer signal origin squamous cell carcinoma (not HPV-associated) includes cancers such as keratinizing and non-keratinizing squamous cell carcinoma of bronchus or lung, HPV-negative squamous cell carcinoma of the larynx, verrucous carcinoma of the urothelial tract, squamous cell carcinoma of the esophagus, basaloid carcinoma of the salivary glands, or some combination thereof. An example cancer that may have a different cancer signal origin includes HPV-positive squamous cell carcinomas, as their cancer signal origin is HPV-associated carcinoma.

In one or more embodiments, the cancer signal origin HPV-associated carcinoma includes cancers such as HPV-positive oropharyngeal squamous cell carcinomas, HPV-positive SCC of the cervix, HPV-positive SCC of the anus, HPV-positive adenocarcinomas of the cervix, or some combination thereof. Example cancers that may have a different cancer signal origin include HPV-negative squamous cell carcinomas of the larynx, squamous cell carcinomas in the H&N region without clinical or molecular evidence of HPV status, squamous cell carcinomas in the cervix or anus with a conclusive negative test result for HPV status, squamous cell carcinoma of the skin in the anal region, non HPV-associated cancers in a patient with an active HPV infection, or some combination thereof.

In one or more embodiments, the cancer signal origin hepatocellular carcinoma includes cancers such as hepatocellular carcinoma and all its subtypes. Example cancers that may have a different cancer signal origin include intrahepatic cholangiocarcinoma, carcinomas in the liver that are not identified as hepatocellular carcinoma, or some combination thereof.

In one or more embodiments, the cancer signal origin neoplasm of Mullerian origin includes cancers such as ovarian serous cystadenocarcinoma, endometrioid adenocarcinoma of ovary or uterus, clear cell carcinoma of ovary or uterus, malignant Mullerian mixed tumor, carcinosarcoma of the uterus, HPV-negative adenocarcinoma of the cervix, or some combination thereof. Example cancers that may have a different cancer signal origin include HPV-positive squamous cell carcinomas of the cervix, cancers reported in ovary or uterus from a different cellular lineage (for example uterine leiomyosarcomas), germ cell carcinomas, or some combination thereof.

In one or more embodiments, the cancer signal origin transitional cell carcinoma includes cancers such as transitional cell carcinoma of the bladder, urothelial carcinoma of the renal pelvis, urothelial carcinoma of the ureter, urothelial or transitional cell carcinoma reported in the kidney, or some combination thereof. Example cancers that may have a different cancer signal origin include adenocarcinomas of the bladder (if not reported as transitional cell carcinoma), cancers of the kidney or renal pelvis not reported as urothelial or transitional cell carcinomas, or some combination thereof.

In one or more embodiments, the cancer signal origin mesenchymal tumor includes cancers such as sarcomas in muscles or connective tissue, uterine leiomyosarcoma, malignant solitary fibrous tumor, osteosarcoma, gastrointestinal stromal tumor, malignant fibrous histiocytoma of skin, malignant Hemangiopericytoma in the brain, or some combination thereof. Example cancers that may have a different cancer signal origin include myeloid sarcoma or other myeloid neoplasm cancers.

In one or more embodiments, the cancer signal origin melanocytic neoplasm includes cancers such as melanoma of skin, mucosal melanoma of the head & neck region, conjunctival and uveal melanoma, amelanotic melanoma, or some combination thereof. Example cancers that may have a different cancer signal origin include Merkel cell carcinoma, sweat gland carcinoma, basal cell carcinoma of skin, squamous cell carcinoma of skin, or some combination thereof.

In one or more embodiments, the cancer signal origin mesothelial neoplasm includes cancers such as pleural mesothelioma, peritoneal mesothelioma, or some combination thereof. An example cancer that may have a different cancer signal origin includes cancers of the pleura that are not of mesothelial origin.

In one or more embodiments, the other category of cancer signal origin includes cancers such as germ cell tumors, anaplastic carcinoma, pleomorphic carcinoma, medullary carcinoma (if not of thyroid), undifferentiated carcinoma, mucoepidermoid carcinoma, astrocytoma, glioblastoma, lymphoepithelial carcinoma, carcinosarcoma (if not of Mullerian origin), or some combination thereof.

In one or more embodiments, some cases may have no cancer signal origin for cancer biology, or an ambiguous cancer signal origin. Cancers in this group include carcinoma NOS, malignant neoplasm NOS, NSCLC NOS, combined hepatocellular carcinoma and cholangiocarcinoma, adenosquamous carcinoma, biphenotypic leukemia, infiltrating duct mixed with other types of carcinoma, cancers with mixed subtypes or collision tumors. However collision tumor of SCLC and adenocarcinoma in the lung or combined small cell carcinomas have the cancer signal origin neuroendocrine tumor or carcinoma, mixed tumors of adenocarcinoma or squamous cell carcinoma and a low-grade neuroendocrine tumor have the cancer signal origin of the epithelial component, and mixed tumors with only one malignant component (for example adenosarcoma) have the target cancer signal origin of that malignant component, other mixed tumors, or some combination thereof.

### IV.B. Training of Parallel CSO Classifiers

An analytics system trains the parallel CSO classifiers. Training generally entails leveraging training data to build a predictive model that accurately predicts the cancer signal origin. The training data may be derived from training samples collected from subjects with varying cancer diagnoses. In some embodiments, the parallel CSO classifiers are deployed in response to detecting a cancer signal in a sample. In such embodiments, the training data for the parallel CSO classifiers may be derived from cancer subjects with varying cancer signal origins. Each cancer signal origin can be characterized by the CSO characteristics of an organ or organ group and a tumor biology type. In one or more embodiments, in training the CSO classifiers, the analytics system may obtain two or more training datasets, wherein at least one of the training datasets includes CSO characteristics for training of one type of CSO classifier. For example, the analytics system may receive a general training dataset usable for training any CSO classifier, and a second specific training dataset usable for training one type of CSO classifier. In another example, the analytics system may leverage a first training dataset specific to one type of CSO classifier and a second training dataset specific to another type of CSO classifier. The training data can be utilized to generate a multiplicity of training sets, with each training set used for training of a CSO classifier. When trained, the analytics system can cross-validate the trained CSO classifiers to validate the predictive accuracy of the CSO classifiers. The trained CSO classifiers are advantageous in that they learn patterns in the training data that are indicative of the various CSO classes. In particular, the parallel training process adds granularity in the CSO prediction by separately predicting organ or organ group from tumor biology. The added granularity provides greater insight to inform work-up steps for the diagnosis of cancer when a cancer signal is detected in a sample.

FIG. 5A is an exemplary flowchart describing a process 500 of training parallel cancer source of origin (CSO) classifiers, according to one or more embodiments. The analytics system may perform the process 500. In other embodiments, another computing device or system may perform any, some, or all of the steps of the process 500. In the embodiment shown in FIG. 5A, the analytics system trains two parallel CSO classifiers, one for predicting CSO organ or organ group and another for predicting CSO tumor biology class.

The analytics system obtains 505 cancer samples for training the parallel CSO classifiers. Each cancer sample is derived from a subject positively diagnosed with cancer. The cancer sample may include methylation sequencing data (e.g., obtained through WGBS or a targeted methylation assay) and CSO labels including a known organ or organ group and a known tumor biology class. In other embodiments, each cancer sample may include additional CSO labels for other CSO characteristics. In some embodiments, the analytics system receives a cancer diagnosis for each cancer sample. From the cancer diagnosis, the analytics system can parse the CSO labels for the various CSO characteristics. For example, the analytics system may, for a cancer sample diagnosed with HPV-positive adenocarcinomas of the cervix, parse out that the cancer signal origin for organ or organ group is cervix and the tumor biology CSO class is HPV-associated carcinoma.

The analytics system, for each cancer sample, generates 510 a feature vector based on the methylation sequencing data. The feature vector may comprise a plurality of methylation features based on the methylation sequencing data. A methylation feature may be a characteristic of the sequencing data related to methylation of fragments in the sequencing data. For example, one type of methylation feature may be methylation density at a particular locus. The methylation density is a percentage of sequence reads that, at the particular locus, have a methylated status. As another example, another type of methylation feature may be density, at a particular locus, of sequence reads that are highly methylated or density, at a particular locus, of sequence reads that are highly unmethylated. In another example, another type of methylation feature may be a count of sequence reads overlapping a particular locus that are identified as anomalously methylated (e.g., as described in FIGS. 3A & 3B). The locus may comprise one or more CpG sites. For example, one locus may be a single CpG site, whereas a second locus may be a string of adjacent CpG sites, i.e., a CpG region.

In one or more embodiments, the feature vector may comprise methylation features spanning the targeted loci. In other embodiments, the analytics system may perform feature selection to identify particularly informative methylation features for CSO classification. Such feature selection may leverage calculating mutual information gain for each methylation feature in discriminating between different classes classified for by the classifier. The features may be ranked according to the information gain and selected accordingly. Discriminatory features are features that contribute to classifying between different labels. The analytics system may evaluate the discriminatory power of features by calculating the information gain as a measure of correlation between the feature and the labels, i.e., a feature with high information gain is more correlated to the labels than a feature with low information gain. The analytics system may identify discriminatory features based on the calculated information gain. In one or more embodiments, the analytics system may select discriminatory features as those with information gain above a threshold, or those with information gain above a certain percentile amongst all features.

In some embodiments, the number of features considered is 100 or more features, 200 or more features, 300 or more features, 400 or more features, 500 or more features, 600 or more features, 700 or more features, 800 or more features, 900 or more features, 1,000 or more features, 1,500 or more features, 2,000 or more features, 2,500 or more features, 3,000 or more features, 3,500 or more features, 4,000 or more features, 4,500 or more features, 5,000 or more features, 6,000 or more features, 7,000 or more features, 8,000 or more features, 9,000 or more features, 10,000 or more features, 15,000 or more features, 20,000 or more features, 25,000 or more features, 30,000 or more features, 35,000 or more features, 40,000 or more features, 50,000 or more features, 75,000 or more features, or 100,000 or more features.

The analytics system generates 515 a first training data set inclusive of the feature vectors of the cancer samples and the known organ or organ group as the CSO class. The first training data set may exclude the tumor biology classes or other CSO characteristics that are in parallel assigned to each training case. As described above, in some embodiments, the analytics system may perform feature selection to identify discriminatory features for use in training of the organ or organ group classifier. Upon feature selection, the analytics system may modify the feature vector to include just the selected features. Such modification of the feature vectors results in a data reduction of the training data set, thereby improving computing efficiency. In some embodiments, the training data set is stored as a data table (or a data array) with each feature vector and known organ type as an entry in the data table.

The analytics system trains 520 the organ type classifier with the first training data set to predict organ or organ group CSO class based on an input feature vector. The analytics system may train the organ or organ group classifier as a computer model comprising a plurality of parameters and a function that relates the input feature vector to the predicted organ or organ group CSO class. In one or more embodiments, the analytics system may train the organ or organ group classifier as a machine-learning model implementing one or more machine-learning algorithms. In further embodiments, the analytics system may train the organ or organ group classifier as a neural network comprising layers of nodes that are interconnected. To perform the training, the analytics system may feed batch(es) of feature vectors through the organ type classifier while adjusting parameters of the organ type classifier in order to steer predictions of the organ type classifier towards the known organ or organ group CSO class of each cancer sample. The analytics system may cross-validate the trained organ or organ group classifier to evaluate the predictive accuracy of the organ type classifier.

In some embodiments, the number of parameters in the organ type classifier is 1,000 or more parameters, 1,500 or more parameters, 2,000 or more parameters, 2,500 or more parameters, 3,000 or more parameters, 3,500 or more parameters, 4,000 or more parameters, 4,500 or more parameters, 5,000 or more parameters, 6,000 or more parameters, 7,000 or more parameters, 8,000 or more parameters, 9,000 or more parameters, 10,000 or more parameters, 15,000 or more parameters, 20,000 or more parameters, 25,000 or more parameters, 30,000 or more parameters, 35,000 or more parameters, 40,000 or more parameters, 50,000 or more parameters, 75,000 or more parameters, 100,000 or more parameters, 200,000 or more parameters, 300,000 or more parameters, 400,000 or more parameters, 500,000 or more parameters, 600,000 or more parameters, 700,000 or more parameters, 800,000 or more parameters, 900,000 or more parameters, 1,000,000 or more parameters, 2,000,000 or more parameters, 3,000,000 or more parameters, 4,000,000 or more parameters, 5,000,000 or more parameters, 6,000,000 or more parameters, 7,000,000 or more parameters, 8,000,000 or more parameters, 9,000,000 or more parameters, or 10,000,000 or more parameters.

The analytics system generates 525 a second training data set inclusive of the feature vectors of the cancer samples and the known tumor biology CSO class. The second training data set may exclude the organ group or other CSO characteristics. As described above, in some embodiments, the analytics system may perform feature selection to identify discriminatory features for use in training of the tumor biology CSO classifier. Upon feature selection, the analytics system may modify the feature vector to include just the selected features. Such modification of the feature vectors results in a data reduction of the training data set, thereby improving computing efficiency. In some embodiments, the training data set is stored as a data table (or a data array) with each feature vector and known organ group and each cancer biology CSO as an entry in the data table. The first training data set and the second training data set are different. While they might contain genetic or epigenetic data from the same samples, the CSO classes are different between the first training data set and the second training data set. For two, feature selection may modify feature vectors of the cancer samples to be different between the first training data set and the second training data set.

The analytics system trains 530 the tumor biology CSO classifier with the second training data set to predict tumor biology CSO class based on an input feature vector. The analytics system may train the tumor biology CSO classifier as a computer model comprising a plurality of parameters and a function that relates the input feature vector to the predicted tumor biology CSO class. In one or more embodiments, the analytics system may train the tumor biology CSO classifier as a machine-learning model implementing one or more machine-learning algorithms. In further embodiments, the analytics system may train the cancer biology CSO classifier as a neural network comprising layers of nodes that are interconnected. To perform the training, the analytics system may feed batch(es) of feature vectors through the tumor biology CSO classifier while adjusting parameters of the tumor biology CSO classifier in order to steer predictions of the tumor biology type classifier towards the known tumor biology CSO class of each cancer sample. The analytics system may cross-validate the trained tumor biology CSO classifier to evaluate the predictive accuracy of the tumor biology type classifier.

In some embodiments, the number of parameters in the tumor biology CSO classifier is 1,000 or more parameters, 1,500 or more parameters, 2,000 or more parameters, 2,500 or more parameters, 3,000 or more parameters, 3,500 or more parameters, 4,000 or more parameters, 4,500 or more parameters, 5,000 or more parameters, 6,000 or more parameters, 7,000 or more parameters, 8,000 or more parameters, 9,000 or more parameters, 10,000 or more parameters, 15,000 or more parameters, 20,000 or more parameters, 25,000 or more parameters, 30,000 or more parameters, 35,000 or more parameters, 40,000 or more parameters, 50,000 or more parameters, 75,000 or more parameters, 100,000 or more parameters, 200,000 or more parameters, 300,000 or more parameters, 400,000 or more parameters, 500,000 or more parameters, 600,000 or more parameters, 700,000 or more parameters, 800,000 or more parameters, 900,000 or more parameters, 1,000,000 or more parameters, 2,000,000 or more parameters, 3,000,000 or more parameters, 4,000,000 or more parameters, 5,000,000 or more parameters, 6,000,000 or more parameters, 7,000,000 or more parameters, 8,000,000 or more parameters, 9,000,000 or more parameters, or 10,000,000 or more parameters.

In one or more embodiments, the analytics system in parallel trains the organ or organ group classifier and the tumor biology classifier. Parallel training refers to separately training of the two models such that parameters are not shared between the two models. Parallel training may also entail utilizing the same base sequencing data, as both models are trained using the same base methylation sequencing data from the cancer samples. In embodiments, however, the features used to train the organ or organ group classifier may differ from the tumor biology classifier. Accordingly, the analytics system, when training each type of classifier, modifies the methylation sequencing data to target the features for the particular type of classifier being trained, i.e., yielding two divergent derivative training datasets.

In other embodiments, the analytics system may sequentially train the organ or organ group classifier and the tumor biology classifier. In sequential training, results of the first classifier may be appended to the feature vector as input to the second classifier. In such embodiments, the second classifier is trained with the added results or known CSO classes of the first classifier. In other embodiments, the analytics system may train multiple CSO classifiers based on the prediction of the first CSO prediction by the first CSO classifier. For example, the first classifier may be the organ or organ group classifier. For each organ or organ group, the analytics system trains a separate tumor biology classifier. So, for example, with 15 organs or organ groups, the analytics system trains 15 tumor biology classifiers. Each tumor biology classifier is trained with cancer samples having the same organ or organ group.

The advantages of separately training the organ or organ group classifier and the tumor biology classifier are multifold. For one, predicting organ or organ group and tumor biology (and/or any other CSO characteristic) separately provides granularity to a CSO prediction compared to a single CSO classifier with confounding classes or CSO classes. The added granularity better informs diagnostic workup options, which can approve the efficiency of a safe workup for a case with cancer signal detected. For two, the analytics system distills two disparate training data sets from the same set of sequencing data. Such distillation compacts the assaying process, while still adding the above-noted granularity to the CSO prediction. For three, parallel training of the CSO classifiers permits each CSO classifier to infer distinct patterns for discriminating between the CSO labels. All such advantages amount to technological improvements to the assaying process and the CSO predictive analyses. Moreover, such advantages improve CSO predictions thereby better informing treatment options, likely resulting in improved treatment outcomes.

### IV.B. Deployment of Parallel Cso Classifiers

The analytics system deploys the trained CSO classifiers. The CSO classifiers may be trained according to the process 500 described above in FIG. 5A. The analytics system may deploy the trained CSO classifiers to output a CSO prediction including one or more CSO characteristics of the sample. For example, the CSO prediction may include an organ or organ group and a tumor biology. The analytics system may deploy the trained CSO classifiers on samples predicted to have cancer or known to have cancer.

FIG. 5B is an exemplary flowchart describing a process 540 of deployment of parallel CSO classifiers, according to one or more embodiments. The analytics system may perform the process 540. In other embodiments, another computing device or system may perform any, some, or all of the steps of the process 540. In the embodiment shown in FIG. 5A, the analytics system deploys two parallel CSO classifiers, one for predicting CSO organ or organ group and another for predicting CSO tumor biology.

The analytics system obtains 545 a test sample comprising methylation sequencing data. In one or more embodiments, the test sample may have an unknown cancer status. In other embodiments, the test sample may have a positive cancer diagnosis. The methylation sequencing data may be derived from sequencing of a biological sample comprising nucleic acid fragments (e.g., via WGBS or a targeted methylation assay).

The analytics system generates 550 a feature vector for the test sample based on the methylation sequencing data. The methylation features may be the same features as described above in FIG. 5A.

In some embodiments, the analytics system applies 555 a cancer classifier to predict cancer status of the test sample. In such embodiments, the test sample may have an unknown cancer status. As such, the cancer classifier (e.g., as trained by the process 400 of FIG. 4A) may be applied to determine a cancer prediction. The cancer prediction may indicate a cancer status of the subject from which the test sample is derived.

The analytics system applies 560 an organ type classifier to predict an organ or organ group for the test sample. In some embodiments, the analytics system may modify the feature vector according to selected features for the organ or organ group classifier, to generate a first reduced feature vector. The analytics system inputs the feature vector (or the first reduced feature vector) into the organ type classifier, which outputs an organ or organ group prediction for the test sample based on the input feature vector. In one or more embodiments, the organ or organ group prediction identifies one of the many organ or organ group classified for as the primary source of origin of the cancer.

The analytics system applies 565 a tumor biology classifier to predict a tumor biology for the test sample. In some embodiments, the analytics system may modify the feature vector according to selected features for the tumor biology classifier, to generate a second reduced feature vector. The analytics system inputs the feature vector (or the second reduced feature vector) into the tumor biology classifier, which outputs a tumor biology prediction for the test sample based on the input feature vector. In one or more embodiments, the tumor biology prediction identifies one of the many tumor biologies classified for.

In some embodiments, the analytics system may apply one CSO classifier after another. In such embodiments, the latter CSO classifier may further input a prediction by the former CSO classifier in conjunction with the feature vector to output a latter prediction. For example, the organ type classifier may output a CSO prediction of an organ or organ group. The analytics system may input the feature vector and the predicted organ type into the tumor biology classifier to predict the tumor biology type. In the converse example, the tumor biology classifier may output a CSO prediction of tumor biology for the sample. The analytics system may then input the feature vector (e.g., which may be specific to the organ or organ group classifier) and the predicted tumor biology into the organ or organ group classifier to predict the organ or organ group of the tumor.

The analytics system may consolidate 570 predictions from the CSO classifiers. The analytics system may receive the CSO prediction from the organ or organ-group classifier together with the CSO prediction from the tumor biology classifier. In this step, the two predictions may be compared against a list of combined CSO predictions that provide information for a safe and efficient diagnostic workup of a detected cancer signal. The analytics system may determine if the organ or organ group prediction and if the cancer biology prediction are reported.

The analytics system reports 575 the results selected in step 570 and, optionally, a diagnostic workup recommendation. The results may include the cancer signal detection (e.g., output by the cancer classifier) and/or the selected CSO prediction (e.g., output by the CSO classifiers). In some embodiments, the results reported by the analytics system can include the cancer signal detection, a basic CSO prediction (e.g. organ system), and additional prediction information from the CSO classifiers such as organ type and/or tumor biology. In some embodiments, the analytics system may identify one or more diagnostic workup options based on the cancer signal detection and/or the CSO predictions. Diagnostic workup options may be associated with various CSO prediction combinations in a database.

With the results and, optionally, the diagnostic workup recommendation, a healthcare provider may consult with the subject to determine a diagnostic workup plan. In some embodiments, the diagnostic workup plan may include one or more of the options recommended by the analytics system. In one or more embodiments, the analytics system may aid in ordering up follow-on diagnostic tests (e.g., in response to directives by the healthcare provider.

### V. Applications

In some embodiments, the methods, analytics systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has a detectable cancer signal.

In one or more embodiments, the likelihood or probability score can be assessed at multiple different time points (e.g., before or after treatment) to monitor disease progression or to monitor treatment effectiveness (e.g., therapeutic efficacy). For example, the individual being monitored may be subject to recurrent sample draws. The biological sample drawn from the individual may be sequenced, e.g., by one or more sequencing devices, to measure sequencing data from the sample. The analytics system may perform various analyses, including any of the cancer classification analyses described elsewhere. In the context of monitoring progression, recurrent sample collection, sequencing, and analysis can track change in cancer signal in the individual over time. If cancer signal is increasing over time, the analytics system may infer the tumor is malignant and progressing. If the cancer signal plateaus, the analytics system may infer the tumor is benign or stagnant. In the context of treatment assessment, the analytics system may collect samples, sequence the samples, and analyze the samples to predict cancer signal before versus after treatment. If the cancer signal decreases following treatment administration, then the analytics system may infer the treatment is successful. The analytics system may further compare the change in cancer signal across a population to assess efficacy individual-to-individual. In one or more embodiments, upon detecting a significant cancer signal, the analytics system may transmit a notification to the individual (e.g., for viewing on their personal computing device) to visit a healthcare provider for diagnostic workup.

In still other embodiments, the likelihood or probability score can be used to make or influence a clinical decision (e.g., diagnostic workup of a cancer signal detection to diagnose a cancer, treatment selection, assessment of treatment effectiveness, etc.). For example, in one embodiment, if the probability score exceeds a threshold, a physician can prescribe an appropriate treatment. In other embodiments, the prediction results may include a CSO prediction comprising one or more predicted CSO classes. For example, the CSO prediction may include an organ or organ group, a tumor biology, a cell of origin, or some combination thereof. Such results (or combination of results) may inform diagnostic workup options, treatment options and follow-up strategies between the healthcare provider and the subject.

### V.A. Detection of Cancer

In some embodiments, the methods and/or classifier of the present invention are used to detect the presence or absence of cancer in a subject unsuspected of having cancer. For example, a classifier (e.g., as described above in Section III) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e., binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of different cancer signal origins being classified (i.e., multiclass classification) for has a prediction value (e.g., scored between 0 and 100). The prediction values may correspond to a likelihood that a given training sample (and during inference, training sample) has each of the cancer signal origins. The analytics system may identify the cancer signal origin that has the highest prediction value and indicate that the test subject likely has a cancer from this signal origin. In other embodiments, the analytics system further compares the highest prediction value to a threshold value (e.g., 50, 55, 60, 65, 70, 75, 80, 85, etc.) to determine that the test subject likely has a cancer of that signal origin. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

In some embodiments, the methods and/or classifier of the present invention are used to determine a cancer source of origin prediction in a subject suspected of having cancer. For example, one or more CSO classifiers (e.g., as described above in Section IV) can be used to determine a cancer source of origin prediction to aid in diagnostic workup options.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### V.B. Parallel Cancer Source of Origin Classification

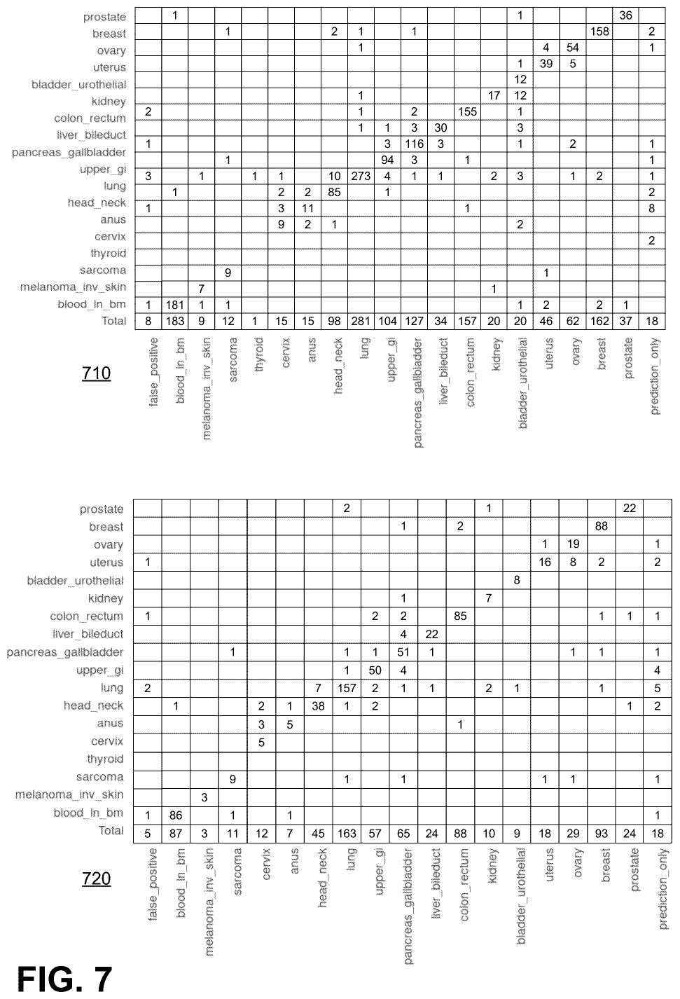

In some embodiments, the methods and/or classifier of the present invention are used to determine multiple separate CSO predictions for a sample. For example, parallel trained CSO classifiers (e.g., as described above in Section IV) can be used to determine a CSO prediction describing an organ or organ group, a tumor biology class, a cell type, or some combination thereof that describe the origin of a cancer signal observed in a sample. In the example implementations with results shown in FIGS. 7 & 8, the organ or organ group classifier and the tumor biology classifier may be trained according to the process 500 of FIG. 5A and deployed according to the process 540 of FIG. 5B. In an example to demonstrate the feasibility to train classifiers separately for organ and organ group and cancer biology signal origin from the same cases and the same genetic or epigenetic information, a training population had 2,496 evaluable study participants with invasive cancer and 2,591 participants without cancer. At 99.4% specificity, 1,401 cancer cases were detected consistently by both classifiers. The first classifier for organ or organ group had 91.8% [90.2-93.2](1,286/1,401) CSO predictions correct. The second classifier for tumor biology was trained independent of the first classifier with the tumor biology classifier prediction correct for 86.2% [84.2-87.9](1,207/1,401). 97 of the 1,401 detected cases had clinical information (for example, “Carcinoma not otherwise specified”) that did not allow to unambiguously assign a target CSO to train a classifier or assess correctness of a prediction, and these were counted as not correct. Both classifiers then made predictions for a held-out population of 1,583 patients with cancer and 521 patients without cancer. 763 cases had cancer signal detected. The first classifier for organ or organ group had 87.9% [85.4-90.2](671/763) CSO predictions correct. The second classifier for tumor biology had a CSO prediction correct for 84.3% [81.5-86.8](643/763).

FIG. 7 illustrates two confusion matrices demonstrating the predictive accuracy of an organ or organ group classifier, according to one or more example implementations. The top confusion matrix 710 illustrates the predictive accuracy from cross-validation with the training cohort. The bottom confusion matrix 720 illustrates the predictive accuracy of a holdout set. For both confusion matrices, the x-axis is the known CSO class (e.g., organ or organ group that represents clinical truth), and the y-axis is the predicted CSO class. Notably, the predictive accuracy is consistent across the two exemplary results.

FIG. 8 illustrates two confusion matrices demonstrating the predictive accuracy of a tumor biology classifier, according to one or more example implementations. The top confusion matrix 810 illustrates the predictive accuracy from cross-validation with the training cohort. The bottom confusion matrix 820 illustrates the predictive accuracy of a holdout set. For both confusion matrices, the x-axis is the known CSO class (e.g., tumor biology representing clinical truth), and the y-axis is the predicted CSO class. Notably, the predictive accuracy is consistent across the two exemplary results.

### V.C. Cancer and Treatment Monitoring

In some embodiments, the cancer prediction can be assessed at multiple different time points (e.g., or before or after treatment) to make a patient prognosis, predict response to a candidate treatment, to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### V.D. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer signal origin) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g., tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V.E. Kit Implementation

Also disclosed herein are kits for performing the methods described above including the methods relating to the cancer classifier. The kits may include one or more collection vessels for collecting a sample from the subject comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In other embodiments, samples collected via the kit are provided to a sequencing laboratory that may use the sequencing panels to sequence the nucleic acids in the sample. The WBC contamination detection may be applied to various configurations of the kit, to minimize WBC contamination potentially originating from components of the kit. For example, experiments may be run comparing types of collection vessels. WBC contamination can be assessed and compared between the types of collection vessels to identify an optimal type that minimizes WBC contamination.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illuminate how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address or QR code which can be used via the internet to access the information at a removed site.

### VI. ADDITIONAL CONSIDERATIONS

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

What is claimed is:

1. A method for training independent parallel cancer signal origin (CSO) classifiers, the method comprising:
obtaining training samples derived from subjects with a known cancer diagnosis, each training sample comprising methylation sequence reads corresponding to nucleic acid fragments in a biological sample collected from each subject and each known cancer diagnosis including a known organ or organ group of a plurality of organs or organ groups affected and a known tumor biology of a plurality of tumor biology classes;
generating, for each training sample, a feature vector based on the methylation sequence reads;
generating a first training data set comprising the feature vectors for the training samples and the known organ or organ group;
training an organ or organ group classifier with the first training data set to predict an organ or organ group from the plurality of organs or organ groups based on an input feature vector;
generating a second training data set comprising the feature vectors for the training samples and the known tumor biology classes; and
training a tumor biology classifier with the second training data set to predict tumor biology from the plurality of tumor biology classes based on input feature vector.

2. The method of claim 1, further comprising:
extracting, for each training sample, the known organ or organ group and the known tumor biology class from the known cancer diagnosis and clinical information.

3. The method of claim 1, wherein the feature vector is based, at least in part, on methylation features based on the methylation sequence reads.

4. The method of claim 3, wherein the methylation features include: methylation density at one or more loci, density of hypermethylated sequence reads at one or more loci, density of hypomethylated sequence reads at one or more loci, a count of methylation sequence reads determined to be anomalously methylated at one or more loci, or some combination thereof.

5. The method of claim 1, wherein generating the first training data set comprises excluding information regarding tumor biology.

6. The method of claim 1, wherein generating the second training data set comprises excluding information regarding the affected organ or organ group.

7. The method of claim 1, further comprising:
determining, for each feature, information gain in discriminating between the organs or organ groups;
identifying discriminatory features for the organ or organ group classifier based on the information gains; and
modifying the feature vectors of the first training set to consist of the discriminatory features, wherein the modified feature vectors are used in training of the organ or organ group classifier.

8. The method of claim 1, further comprising:
determining, for each feature, information gain in discriminating between the tumor biology classes;
identifying discriminatory features for the tumor biology classifier based on the information gains; and
modifying the feature vectors of the second training set to consist of the discriminatory features, wherein the modified feature vectors are used in training of the tumor biology classifier.

9. The method of claim 1, wherein the organ or organ group classifier or the tumor biology classifier are machine-learning models.

10. The method of claim 1, further comprising training the organ or organ group classifier and the tumor biology classifier in parallel training processes.

11. The method of claim 1, further comprising training the organ or organ group classifier prior to training the tumor biology classifier.

12. The method of claim 11, wherein outputs of the organ or organ group classifier are appended to the feature vectors of the second training data set prior to training of the tumor biology classifier.

13. The method of claim 1, further comprising training the tumor biology classifier prior to training the organ or organ group classifier.

14. The method of claim 13, wherein outputs of the tumor biology classifier are appended to the feature vectors of the first training data set prior to training of the organ or organ group classifier.

15. The method of claim 1, wherein the organs or organ groups include: breast; prostate; lung; head or neck; anus; cervix; ovary or fallopian tubes; uterus; bladder or urothelial; kidney; stomach or esophagus; liver or intrahepatic bile duct; pancreas, extrahepatic bile duct, or gall bladder; colon or rectum; bone or soft tissue; skin; blood, lymphatic system, or bone marrow; thyroid; ambiguous tissue; or some combination thereof.

16. The method of claim 1, wherein the tumor biology classes include: lymphoid neoplasm, myeloid neoplasm, plasma cell neoplasm, neuroendocrine carcinoma or tumor, adenocarcinoma, squamous cell carcinoma and not human-papillomavirus-associated (HPV-associated), HPV-associated carcinoma, hepatocellular carcinoma, neoplasm of Mullerian origin, transitional cell carcinoma, mesenchymal tumor, melanocytic neoplasm, mesothelial neoplasm, other tumor biology, ambiguous tumor biology, or some combination thereof.

17. A method for predicting cancer signal of origin (CSO), the method comprising:
obtaining a test sample derived from a subject, the test sample comprising methylation sequence reads corresponding to nucleic acid fragments in a biological sample collected from the subject;
generating, for the test sample, a first feature vector based on the methylation sequence reads associated with a first set of features identified as discriminatory for organ or organ group classification;
generating, for the test sample, a second feature vector based on the methylation sequence reads associated with a second set of features identifies as discriminatory for tumor biology classification;
applying an organ or organ group classifier to the first feature vectors to predict an organ or organ group of a cancer associated with the test sample from a plurality of organs or organ groups;
applying a tumor biology classifier to the second feature vector to predict a tumor biology for the cancer associated with the test sample from a plurality of tumor biology classes;
wherein the organ or organ group classifier and the tumor biology classifier are independently trained on training samples derived from subjects with a known cancer diagnosis including a known organ or organ group of a plurality of organs or organ groups affected and a known tumor biology of a plurality of tumor biology classes, each training sample comprising methylation sequence reads corresponding to nucleic acid fragments in a biological sample collected from each subject; and
informing a diagnostic workup to diagnose a cancer based on the predicted organ or organ groups and the predicted tumor biology.

18. The method of claim 17, wherein the organ or organ group classifier and the tumor biology classifier are trained by:
generating, for each training sample, a feature vector based on the methylation sequence reads of the training sample;
generating a first training data set comprising the feature vectors for the training samples and the known organ or organ group of the known cancer diagnosis;
training an organ or organ group classifier with the first training data set to predict an organ or organ group from the plurality of organs or organ groups based on an input feature vector;
generating a second training data set comprising the feature vectors for the training samples and the known tumor biology classes of the known cancer diagnosis; and
training a tumor biology classifier with the second training data set to predict tumor biology from the plurality of tumor biology classes based on input feature vector.

19. The method of claim 18, wherein generating the first training data set comprises excluding information regarding tumor biology, and wherein generating the second training data set comprises excluding information regarding the affected organ or organ group.

20. A method for providing a report for a test sample for a patient to assist with a diagnostic workup of the patient, the report comprising a cancer signal detected readout and a cancer signal origin (CSO) prediction, the CSO prediction comprising a predicted organ or organ groups for the CSO and a predicted tumor biology for the CSO, the method comprising:
obtaining the test sample derived from a patient, the test sample comprising methylation sequence reads corresponding to nucleic acid fragments in a biological sample collected from the patient;
generating, for the test sample, a first feature vector based on methylation information selected to be informative of a cancer signal associated with the test sample;
generating, for the test sample, a second feature vector based on the methylation sequence reads associated with a first set of features identified as discriminatory for organ or organ group classification;
generating, for the test sample, a third feature vector based on the methylation sequence reads associated with a second set of features identifies as discriminatory for tumor biology classification;
applying a cancer signal classifier to the first feature vector to predict the cancer signal of a cancer associated with the test sample;
applying an organ or organ group classifier to the second feature vector to predict an organ or organ group of the cancer associated with the test sample from a plurality of organs or organ groups;
applying a tumor biology classifier to the third feature vector to predict a tumor biology for the cancer associated with the test sample from a plurality of tumor biology classes;
wherein the cancer signal classifier is trained on training samples derived from a plurality of cancer-positive subjects and cancer-negative subjects, each cancer-positive subject having a labeled cancer diagnosis and each cancer-negative subject known to not have cancer, each training sample comprising methylation sequence reads corresponding to nucleic acid fragments in a biological sample collected from each subject;
wherein the organ or organ group classifier and the tumor biology classifier are independently trained on training samples derived from subjects with a known cancer diagnosis including a known organ or organ group of a plurality of organs or organ groups affected and a known tumor biology of a plurality of tumor biology classes, each training sample comprising methylation sequence reads corresponding to nucleic acid fragments in a biological sample collected from each subject;
generating the report for the test sample comprising the cancer signal detected readout and the cancer signal origin (CSO) prediction based on results from the cancer signal classifier, the organ or organ type classifier, and the tumor biology classifier, the CSO prediction comprising the predicted organ or organ groups for the CSO and the predicted tumor biology for the CSO; and
providing the report to the patient or a health care provider for the patient.
